(19) United States
(12) Patent Application Publication
(10) Pub. No.: US 2022/0403363 A1
(43) Pub. Date: Dec. 22, 2022

(54) ADAPTER SYSTEM FOR NONRIBOSOMAL PEPTIDE SYNTHETASES AND POLYKETIDE SYNTHASES

(71) Applicant: JOHANN WOLFGANG GOETHE-UNIVERSITÄT FRANKFURT AM MAIN, Frankfurt am Main (DE)

(72) Inventors: Helge BODE, Oberursel (DE); Kenan BOZHÜYÜK, Frankfurt am Main (DE); Jonas WATZEL, Mainz (DE)

(73) Assignee: JOHANN WOLFGANG GOETHE-UNIVERSITÄT FRANKFURT AM MAIN, Frankfurt am Main (DE)

(21) Appl. No.: 17/776,213

(22) PCT Filed: Nov. 12, 2020
(86) PCT No.: PCT/EP2020/081925

§ 371 Date: May 11, 2022

(30) Foreign Application Priority Data
Nov. 12, 2019 (EP) 19208603.1

(51) Int. Cl. C12N 9/00; C12N 15/63; C07K 14/00

(52) U.S. Cl. CPC C12N 9/93; C12N 15/63; C07K 14/00; C07K 2319/70

(57) ABSTRACT

The invention relates to a system for expressing nonribosomal peptide synthetases (NRPSs), polyketide synthases (PKS) or NRPS/PKS hybrid synth(et)ases. NRPS, PKS or hybrids thereof are large multi-domain proteins or multi-domain complexes, the expression of which for the production of peptides often causes difficulties. The invention correspondingly relates to a system for expressing portions of the enzymes which can be assembled post-translationally via protein-protein interactions, introduced in a targeted manner, to form multi-enzyme complexes. The invention discloses protein fragments of such an assembly, and the nucleic acids coding therefor. The invention also relates to a vector system for the protein fragments of the invention and its use for producing functional NRPS/PKS enzyme complexes.

### FIELD OF THE INVENTION

The invention relates to a system for the expression of non-ribosomal peptide synthetases (NRPSs), polyketide synthases (PKS) or NRPS/PKS hybrid synth(et)ases. NRPS, PKS or their hybrids are large multidomain proteins or multidomain complexes whose expression often causes difficulties for the production of peptides. Accordingly, the invention relates to a system for expressing fragments of enzymes which can be assembled post-translationally to form functional multienzyme complexes via specifically introduced protein-protein interactions. The invention discloses protein fragments of such a kit as well as their coding nucleic acids. Also disclosed is a vector system of the protein fragments of the invention and its use for preparing functional NRPS/PKS enzyme complexes.

### DESCRIPTION

Non-ribosomal peptides (NRPs) are peptides produced by non-ribosomal peptide synthetases (NRPSs) with a large structural diversity, which are characterised in particular by cyclic, brachiated or other complex primary structures (Caradec et al., 2014; Caboche et al., 2010). Due to their structural complexity, many of these molecules exhibit therapeutic properties which include antibiotic, immunosuppressive or anticarcinogenic modes of action (Finking and Marahiel, 2004; Felnagle et al., 2008). As a result, they are not only a source for new medicaments, but also serve as basic structures for pharmaceutical reagents (Cane et al., 1998). For this purpose, the natural substances are usually chemically modified by semisynthesis, a process that combines biosynthesis and organic synthesis (Kirschning and Hahn, 2012). Alternatively, novel NRPs can also be produced by reprogramming the NRPSs responsible for the synthesis. For this purpose, the modular structure of the synthetases is often exploited and genetically engineered. Some examples are known in the literature where the reprogramming of NRPSs was successful (Schneider et al., 1998; Chiocchini et al., 2006). Nevertheless, the productivity of these synthetases is usually severely limited (Suo, 2005).

The structural and functional diversity of NRPs is a result of the incorporation of D-amino acids (AS), heterocyclic elements or N-methylated side chains as well as the addition of fats, sugars and halogens (Hur et al., 2012). Examples of NRPs having such structural peculiarities are, for example, bacitracin and vibriobactin, which carry heterocyclic rings, or cyclosporin A and tyrocidine A, which are characterised by the incorporation of D-AS. Daptomycin, on the other hand, is an acetylated peptide which, because of its fatty acid, has a strong antibacterial effect; whereas balhimycin and syringomycin are examples of halogenated NRPs that have antibacterial and antifungal properties.

SYNZIPs are heterospecific synthetic coiled coils that enable controlled protein interaction and are used in synthetic biology. Coiled coils are generally composed of two, three or four amphipathic 20-50 AS-long α helices which form an intertwined left-handed supercoil. They are structural motifs of many proteins and also occur, for example, in the leucine zipper regions of the human bZIP transcription factor. Coiled coils are characterised by a heptade pattern (abcdefg)ₙ which carry hydrophobic AS at positions a and d, while electrostatic AS usually occur at positions e and g. In an α-helical secondary structure, these hydrophobic AS interact with one another and form a narrow hydrophobic interface (Lumb et al., 1994).

SYNZIPs were originally developed for the heterospecific interaction with leucine zipper regions of human bZIP transcription factors (TF). For this purpose, 48 artificial peptides were constructed on the basis of a computer, which were subsequently investigated with regard to their interaction with these peptides (Grigoryan et al., 2009). In a further study, on the other hand, the interaction of the peptides with one another was also tested. To this end, Reinke et al. carried out a protein-microarray assay in which all 48 artificial as well as 7 further coiled-coils of human bZIPs were tested against one another (FIG. 1). From the results of the assay, 27 pairs, 23 synthetic ones (namely SYNZIP 1-23) and three human bZIP structures were selected which showed a strong heterospecific and, at the same time, low homospecific interaction. As indicated in FIG. 1A, the peptides are involved in at least one to a maximum of seven interactions and in some cases can form different networks (FIG. 1 B). Examples of these are linear, annular, branched and orthogonal networks (Thompson et al., 2012). Furthermore, it was concluded from the Asn-Asn pairing on the a-a′ positions that most pairs must be parallel heterodimers (Reinke et al., 2010).

The international publication WO 2019/138117 describes a system for assembling and modifying NRPS. The system uses novel, precisely defined building blocks (units) which comprise condensation subdomains. This strategy enables the efficient combination of assemblies, which are referred to as eXchange Units (XU2.0), irrespective of their naturally occurring specificity for the subsequent NRPS adenylation domain. The system of WO 2019/138117 enables the simple assembly of NRPS with an activity for the synthesis of a peptide with any amino acid sequence, without restrictions due to naturally occurring NRPS units. The system also makes it possible to exchange natural NRPS building blocks with the XU2.0 according to the invention, as a result of which modified peptides are produced. Although the system allows a simple combination of XU2.0 units, it still requires the expression of the assembled NRPS protein in an open reading frame (ORF). This leads to problems, especially in the case of longer NRPs.

It is therefore the purpose of the present invention to produce NRPS modules or submodules (or domains) such as, for example, XU2.0 units from WO 2019/138117, efficiently and flexibly recombinant.

### BRIEF DESCRIPTION OF THE INVENTION

In general and by means of a brief description, the main aspects of the present invention can be described as follows:

In a first aspect, the invention relates to a protein or a protein fragment comprising at least a first domain or partial domain of a non-ribosomal peptide synthetase (NRPS), a polyketide synthase (PKS) or an NRPS/PKS hybrid synth(et)ase (first PKS-NRPS domain), wherein the protein or the protein fragment has an N-terminus or a C-terminus comprising a first binding domain and wherein this first binding domain preferably represents the N-terminus or C-terminus, respectively, of the protein or the protein fragment, and wherein the first binding domain is characterised by the property of being able to enter into a specific protein-protein binding with at least one corresponding second binding domain.

In a second aspect, the invention relates to an isolated nucleic acid construct comprising a first coding region which has a nucleic acid sequence which codes for a protein or protein fragment of the first aspect.

In a third aspect, the invention relates to a vector system for producing a functional NRPS or PKS, wherein the vector system comprises at least one nucleic acid construct according to the second aspect, and wherein the at least one nucleic acid construct is suitable for expressing at least two proteins or protein fragments according to the first aspect, and wherein the at least two proteins or protein fragments are different and together form a functional NRPS, PKS or NRPS/PKS hybrid.

In a fourth aspect, the invention relates to a method for producing a functional (complete) NRPS or PKS, comprising bringing at least a first protein or protein fragment according to the first aspect into contact with a second protein or protein fragment according to the first aspect, wherein the first protein or protein fragment has a terminal first binding domain, and wherein the second protein or protein fragment has the terminal second binding domain instead of the terminal first binding domain.

### DETAILED DESCRIPTION OF THE INVENTION

The elements of the invention are described below. These elements are described with specific embodiments. However, it goes without saying that the elements of the invention can be combined with one another in any manner and in any number in order to obtain additional embodiments. The variously described examples and preferred embodiments should not be interpreted as restricting the present invention only to the explicitly described embodiments or examples. The present disclosure should be understood as describing and including embodiments that combine two or more of the explicitly described embodiments or elements with one another, or that combine one or more of the explicitly described embodiments with any number of the disclosed and/or preferred elements. In addition, all permutations and combinations of all elements described in this application should be regarded as disclosed by the description of the present application, unless the context or technical context otherwise indicates or permits.

The term “partial domain” or “partial C or C/E domain” or “partial domain”, or similar terms, refers to a nucleic acid sequence encoding an NRPS-PKS domain or a protein sequence thereof which is incomplete (not in full length). In this context, the term is to be understood as meaning that, compared with the full-length domain, the partial domain has a contiguous proportion of at least 20%, preferably 30% or 40% or more, of a successive sequence of the full-length domain. A partial domain therefore has a very high degree of sequence identity (90% and more) with respect to a coherent portion (at least 20%, preferably 30% or 40% or more) of the sequence of the full-length domain. For example, the expression describes a C or C/E domain sequence which does not comprise both donor and acceptor sites of an NRPS-C or C/E domain. Partial domains of NRPS have, for example, a sequence length of 100 or more, 150 or more, or about 200 amino acids.

“Compilation” refers to a number of domains. A plurality of NRPS/PKS assemblies includes a complete NRPS-PKS. One or more polypeptides may comprise a module. Module combinations then catalyse longer peptides in combination. In one example, a module may comprise a C domain (condensation domain), an A domain (adenylation domain), and a peptidyl carrier protein domain.

Further structural information on A domains, C domains, didomains, domain-domain interfaces and complete modules can be found at Conti et al. (1997), Sundlov et al. (2013), Samel et al. (2007), Tanovic et al. (2008), Strieker and Marahiel (2010), Mitchell et al. (2012) and Tan et al. (2015).

“Initiation module” means an N-terminal module that can transfer a first monomer to another module (e.g., an extension or final module). In some cases, the further module is not the second module, but one of the C-terminally following modules (for example in the case of the Nocardicin NRPS). In the case of an NRPS, an initiation module comprises, for example, an A (adenylation) domain and a PCP (peptidyl carrier protein) or a T (thiolation) domain. The initiation module can also contain a starter C domain and/or an E domain (epimerisation domain). With a PKS, a possible initiation module consists of an AT domain (acetyl transferase) and an ACP domain (acyl carrier protein). Initiation modules are preferably located at the amino terminus of a polypeptide of the first module of an “assembly series”. Each assembly series preferably contains an initiation module.

The term “extension module” or “elongation module” refers to a module that adds a donor monomer to an acceptor monomer or an acceptor polymer, thereby extending the peptide chain. An elongation module may comprise a C (condensation), Cy (heterocyclisation), E, C/E, MT (methyltransferase), A-MT (combined adenylation and methylation domain), Ox (oxidase) or Re (reductase) domain; an A domain; or a T domain. An elongation domain may further comprise additional E, Re, DH (dehydration), MT, NMet (N-methylation), AMT (aminotransferase) or Cy domains. In addition, an elongation module could be of PKS origin and could comprise the respective domains (ketosynthase (KS), acyltransferase (AT), ketoreductase (KR), dehydratase (DH), enoyl reductase (ER, thiolation (T)).

“Termination module” refers to a module that releases or decouples the molecule (e.g., an NRP, a PK, or combinations thereof) from the assembly series. The molecule can be released, for example, by hydrolysis or cyclisation. Terminating modules may comprise a TE (thioesterase), Cterm (terminal C domain) or Re domain. The termination module is preferably located at the carboxy terminus of an NRPS or PKS polypeptide. The termination module may further comprise additional enzymatic activities (e.g., oligomerase activity).

“Domain” means a polypeptide sequence or a fragment of a larger polypeptide sequence having one or more specific enzymatic activities (i.e., C/E domains have a C and an E function in a domain or another conserved function (i.e., as a binding function for an ACP or T domain). Thus, a single polypeptide may comprise multiple domains. Multiple domains can form modules. Examples of domains are C (condensation), Cy (heterocyclisation), A (adenylation), T (thiolation), TE (thioesterase), E (epimerisation), C/E (condensation/epimerisation), MT (methyltransferase). Ox (oxidase), Re (reductase), KS (ketosynthase), AT (acyltransferase), KR (ketoreductase), DH (dehydratase) and ER (enoyl reductase).

“Non-ribosomally synthesised peptide”, “non-ribosomal peptide” or ‘NRP’ refers to any polypeptide that is not produced by a ribosome. NRPs may be linear, cyclic or branched, and may contain proteinogenic, natural or non-natural amino acids, or any combination thereof. The NRPs include peptides which are produced in a type of assembly line or series (=modular character of the enzyme system, which enables the gradual addition of building blocks to the end product).

“Polyketide” refers to a compound which comprises a plurality of ketone units.

“Non-ribosomal peptide synthetase” or “non-ribosomal peptide synthetase” or “NRPS” refers to a polypeptide or a series of interacting polypeptides which produce a non-ribosomal peptide and can thus catalyse the formation of peptide bonds without ribosomal components. “Polyketide synthase” (PKS) refers to a polypeptide or a series of polypeptides that produce a polyketide without ribosomal components.

“Non-ribosomal peptide synthetase/polyketide synthase hybrid” or “hybrid of non-ribosomal peptide synthetases and polyketide synthases” or “NRPS/PKS hybrid” or “hybrid of NRPS and PKS” or “hybrid of PKS and NRPS” and other corresponding expressions refer to an enzyme system comprising any domains or modules of NRPS and PKS. Such hybrids catalyse the synthesis of natural hybrid substances.

“Change in a structure” means any change in a chemical (e.g., covalent or non-covalent) bond compared to a reference structure.

“Mutation” refers to a change in the nucleic acid sequence, so that the amino acid sequence encoded by the nucleic acid sequence has at least one amino acid change in comparison with the naturally occurring sequence. The mutation can, without limitation, be an insertion, deletion, frame shift mutation or a missense mutation. This term also describes a protein which is encoded by the mutated nucleic acid sequence.

A “variant” is a polypeptide or polynucleotide having at least 10%, 20%, 30%, 40%, 50%, 60%, 70%, 80%, 90%, 95% or 99% sequence identity to a reference sequence. The sequence identity is typically measured using sequence analysis software (for example, sequence analysis software package from the Genetics Computer Group, Biotechnology Center of the University of Wisconsin, 1710 University Avenue, Madison, Wis., USA). 53705, programs: BLAST, BESTFIT, gap or PILEUP/PRETTYBOX). This type of software adapts identical or similar sequences by assigning degrees of homology to various substitutions, deletions and/or other modifications (substitution/scoring matrix: e.g., PAM, Blosum, GONET, JTT).

In a first aspect, the invention relates to a protein or a protein fragment comprising at least a first domain or partial domain of a non-ribosomal peptide synthetase (NRPS), a polyketide synthase (PKS) or an NRPS/PKS hybrid synth(et)ase (first PKS-NRPS domain), wherein the protein or the protein fragment has an N-terminus or a C-terminus comprising a first binding domain and wherein this first binding domain preferably represents the N-terminus or C-terminus, respectively, of the protein or the protein fragment, and wherein the first binding domain is characterised by the property of being able to enter into a specific protein-protein binding with at least one corresponding second binding domain.

A protein or protein fragment, in the sense of this disclosure, is preferably a polypeptide comprising an amino acid sequence which has a high sequence identity to a contiguous portion of an NRPS/PKS, and one or more modules thereof, as well as protein parts thereof.

In the context of this application, the term “binding domain” is intended to denote a polypeptide element, domain or sequence which has the ability to form specific or non-specific covalent or non-covalent bonds with other polypeptide sequences. In a particular embodiment, the second binding domain is an endogenous NRPS/PKS sequence which enables an interaction, for example, and preferably with a SYNZIP according to the invention. Binding domains which enter into a specific binding interaction with other corresponding binding domains are preferred for the purposes of the present invention. In the context of the present invention, these are also referred to as protein interaction domains (PID). For example, these can be polypeptide domains which are responsible for the homo- or heterodimer formation of protein. These kinds of domains are, for example, coiled-coil domains, CH3 domains and leucine zipper domains. Particular preference is given to the so-called SYNZIP domains.

Coiled-coil-protein interaction domains are known in the art. Several non-limiting embodiments of computer programs for creating such PIDs include SOCKET (e.g., as described in Walshaw & Woolfson, J. Mol. Gen. Biol, 2001; 307 (5), 1427-1450, available on the website of the Woolfson Group at the University of Bristol), COILS (e.g., as described in Lupas et al., Science. 1991; 252: 1162-1164 and incorporated by reference into this disclosure), obtainable from the ch. EMBnet.org website), PAIRCOIL (e.g., as in Berger et al., Proc Natl. Acad. Sci. UNITED STATES OF AMERICA. 1995; 92, 8259-8263, available from the groups, csail.mit.edu/cb/paircoil/cgi-bin/paircoil.cgi and MULTICOIL (described, for example, by Wolf et al., Protein Sci. 1997; 6: 1179-1189, available from the group csail.mit. edu/cb/multicoil/cgi-bin/multicoil cgi website.

In some embodiments, the PIDs which form coiled coils are those which are described in Table I by Müller et al., Methods Enzymol. 2000; 328, 261, which are incorporated in this disclosure in their entirety by reference. For example, PIDs that form coiled coils comprise leucine zippers (e.g., as in the proteins GCN4, Fos, Jun, C/EBP and variants or mutants thereof), the peptide “Velcro” (e.g., as described by O'Shea et al., Curr Biol. 1993; 3(10): 658-67), E-Coil/K-Coil (e.g., as described by Tripet et al., Protein Eng. 1996; 9, 1029) and WinZip-A2 and WinZip-B1 (e.g., as described by Arndt et al.), Structure. 2002; (9): 1235-48).

In some embodiments, the PIDs that form coiled coils are heterospecific synthetic coiled coil peptides called SYNZIPs, for example, SYNZIPs 1-22. Detailed information on the SYNZIPs 1-22 is disclosed to Thompson K E, et al: “SYNZIP protein interaction toolbox: in vitro and in vivo specifications of heterospecific coiled-coil interaction domains.” (ACS Synth Biol. 2012 Apr. 20; 1(4): 118-29.); the document is incorporated in this disclosure by reference in its entirety. In some embodiments, the PIDs that are either C or N terminally fused to an NRPS-PKS domain or subdomain are SYNZIP 17 (NEKEELKSKKKAELRNRIEQLKQKREQLKQKIANLRKEIEAYK, SEQ ID NO: 1) and/or SYNZIP 18 (SIAATLENDLARLENARLEKDIANLAKLEREEAYEAYEAYEF, SEQ ID NO: 2). Other combinations of SYNZIPs which can be used in the context of the present invention as a pair of binding domains are listed in the matrix of FIG. 1.

In some embodiments, the PIDs taken into account by the present disclosure include those disclosed on the website of Dr Tony Pawson at Mount Sinai Hospital, Toronto. For example, PIDs include 14-3-3 domains, ADF domains, ANK repeats, ARM repeats, the bar domain of amphiphysin, the BEACH domain, Bcl-2 homology domains (BH) (e.g., BH1), BH2, BH3, BH4), BIR domains, BRCT domains, bromodomains, BTB/POZ domains, CI domains, C2 domains, caspase recruitment domains (CARDs), lymphoid myeloid (CALM) domains with clathrin assembly, calponin homology (CH) domains, chromatin organisation modifier (CHROMO/Chr) domains, CUE domains, death (DD) domains, death effector (DED) domains, DEP domains, Dbl homology (DH) domains, EF hand (EFh) domains, Eps15 homology (EH) domains, epsin NH2-terminal homology (ENTH) domains, Ena/Vasp-homology domain 1 (EVH1 domains), Fox-Box domains, FERM domains, FF domains, formin homology domains 2 (FH2), Forkhead associated domains (FH), FYVE (Fab-1, YGLo23-, Vps27-dn EEA1 domains, GAT- (GGA- and Toml) domains, Gelsolin/Severin/Villin homology (GEL) domains, GLUE from gram-like ubiquitin binding domains in EAP45) domains, GRAM (from glucosyltransferases, Rab-like domains GTPase activators and myotubularin domains, GRIP domains, glycine-tyrosine-phenylalanine domains (GYF), HEAT domains (from Huntington, elongation factor 3, PR65/A, TOR), HECT domains (from homologous to the E6-AP carboxyl-terminus domains), IQ domains, LIM domains, leucine-rich repeat domains (LRR domains), malignant brain tumour domains (MBT domains), Mad homology 1 domains (MH1 domains), MH2 domains, MIU domains (from motif interacting with ubiquitin), NZF domains (Npl4 zinc finger) domains, PAS domains (Per-ARNT Sim domains), Phox and Beml domains (PM domains), PDZ domains (from postsynaptic density 95; PS5-85, large slices, Dig; Zonula occludens-1, ZO-1) ns, Pleckstrin homology domains (PH domains), Polo Box domains, Phosphotyrosin binding domains (PTB domains), Pumilio domains (Puf domains), PWWP domains, Phox homology domains (PX domains), RGS domains (regulator of G protein signalling), RING finger domains, SAM domains (sterile Alpha motive), shade chromo domains (CSD or SC domains), Src-homology-2 domains (5H2 domains), Src homologie-3 domains (SH3 domains), SOCS domains (Cytokin signalling pathway suppressors), SPRY domains, START (of steroidogenic acute regulatory protein (StAR) related lipid transfer) domains, SWIRM domains, Toll/11-1 receptor (TIR) domains, tetratricopeptide repeat (TPR) motif domains, TRAF domains, SNARE domains (of soluble NSF binding protein receptors (SMAP receptors)) (e.g., T-SNARE), Tubby domains, Tudor domains, ubiquitin-associated domains (UBA), UEV domains (ubiquitin E2 variant), ubiquitin interacting motif (UIM) domains, beta domains of Hippel-Lindau tumour suppressor protein (VHLP), VHS domains (of Vps27p, Hrs and STAM), WD40 repeat domains and WW domains.

PIDs can be linked with or without a linker to the C or N terminus of the protein or protein fragment according to the invention. It goes without saying that any PIDs and any linkers may be compatible with aspects of the invention. In some embodiments, the linker is flexible. The linker can be composed of amino acids. In some embodiments, the linker consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 amino acids. In some embodiments, the linker consists of 5-7 amino acids. In some embodiments, the linker is, for example, a Gly-Ser linker.

The protein or protein fragment according to the invention preferably comprises a SYNZIP as first and/or second binding domain, wherein the SYNZIP is selected from a SYNZIP 1-23, preferably from SYNZIP 1, 2, 17, 18 or 19. In some embodiments, preference is given to a protein or protein fragment, wherein the terminus which is opposite the first binding domain comprises a third binding domain, and wherein the third binding domain is characterised by the property of being able to enter into a specific protein-protein binding with at least one corresponding fourth binding domain. Preferably, the first and second binding domains cannot bind to the third and fourth binding domains. According to the invention, NRPS/PKS proteins which consist of three or more individual polypeptides (proteins or protein fragments according to the invention) can be produced with such a structure.

In preferred embodiments, the SYNZIP sequences from Thompson K E, et al. can have a shortening at the C- and/or N-terminus in comparison to the original sequences. This shortening is ideally located at the N- and/or C-terminus, i.e., compared to the original sequence, amino acids are removed at the N- or C-terminus, and the remaining sequence does not change. In preferred embodiments, at least 7, preferably at least 10, amino acids of the SYNZIP always remain. In the case of the SYNZIP pairs used in the context of the invention, either both or else only one SYNZIP can be present in shortened form in accordance with the invention.

The shortening preferably relates to 1 to 15 amino acids and can occur at the C- and/or N-terminus. Shorter SYNZIPs bring a compound NRPS closer to the natural configuration, since the connection point in the normal context comprises considerably fewer amino acids. Furthermore, the shortening is preferably 1 to 10 amino acids long. Thus, for example, the shortening of the N-terminal of the NRPS SYNZIP can affect sequence 9 amino acids, while the shortening of the C-terminal of the NRPS SYNZIP comprises sequence 2 amino acids. This is illustrated in FIG. 16 for the SYNZIP pairs SZ1 and SZ2 or SZ19 and SZ2. However, the shortening of the SYNZIP sequences must not lead to a loss of the SYNZIP pair's pairing property.

Preferably, the first and second binding domains can specifically enter into a bond with the third or fourth binding domain, so that mixtures of peptides can be formed.

The term “terminus” in connection with a protein or protein fragment refers to the respective end of the amino acid polymer. The free amino end is referred to as the “N-terminus”, the free carboxy end as the “C-terminus”. If a feature, for example a sequence or domain or other elements, are arranged at the N- or C-terminus, this means that the corresponding feature makes up the last N-terminal or C-terminal portion of the entire protein and thus represents the N- or C-terminus.

The protein or protein fragment according to the invention in a particular embodiment further comprises at least one, preferably two, three or four or more, further PKS and/or NRPS domain(s), wherein the further PKS and/or NRPS domain(s) is/are arranged in a direct functional arrangement next to the first PKS-NRPS domain. Direct functional arrangement means that the domains can carry out an NRPS/PKS synthesis of a peptide or of a peptide/polyketide spatially.

In a preferred embodiment, the protein or protein fragment does not comprise the at least one corresponding second binding domain. In this embodiment, the second binding domain is located in a second protein or protein fragment according to the invention, wherein the first and second, preferably also other, protein or protein fragment according to the invention then form a group of proteins or protein fragments according to the invention. This group of proteins or protein fragments according to the invention are designed in such a way that a functional NRPS or PKS, or hybrids thereof, can be assembled post-translationally by specific or non-specific binding by means of the binding domains.

In a preferred embodiment of the invention, the first binding domain is arranged at the terminal end in such a way that a specific or non-specific mediated interaction/binding to a corresponding second binding domain is possible under normal conditions. In this case, the second binding domain would be found in a second protein or protein fragment according to the invention. In this case, the composition of NRPS/PKS domains would differ in the first and second protein or protein fragment of the invention.

The first PKS-NRPS domain, or partial domain, according to the invention is selected from any NRPS and/or PKS domain known to the expert. These are preferably selected from an A domain, a C domain, a C/E domain, an E domain, a Cₛₜₐᵣₜ domain, an FT domain, or a T domain. In preferred embodiments, the protein or protein fragment of the invention comprises at least one A domain, a C domain and a T domain, preferably where the protein or protein fragment has at least one NRPS-PKS, initiation module, elongation module or termination module.

The protein or protein fragment of claim 12 or 13, wherein the third binding domain is coupled to the protein or protein fragment via a linker sequence.

The protein or protein fragment of any preceding claim, wherein the binding of the first binding domain to the second binding domain is a non-covalent binding.

In a second aspect, the invention relates to an isolated nucleic acid construct comprising a first coding region which has a nucleic acid sequence which codes for a protein or protein fragment of the first aspect.

The term “nucleic acid” means natural, semisynthetic or completely synthetic as well as modified nucleic acid molecules consisting of deoxyribonucleotides and/or ribonucleotides, and/or modified nucleotides such as “peptide nucleic acids” (PNA), “locked nucleic acids” (LNA) or “phosphorothioates”. Other modifications of the internucleotide phosphates and of the ribose or sugar components may also be present.

A so-called “coding region” refers to a sequence element within a nucleic acid construct of the invention which codes for an expressible protein according to the genetic code.

In one embodiment of the nucleic acid construct of the invention, the first coding region is functionally linked to an expression promoter. An expression promoter denotes a nucleic acid element which is necessary for initiating an RNA transcription and is preferably sufficient. These kind of elements are known to experts. Depending on the expression system, these types of promoters can be selected.

In a further embodiment, the nucleic acid construct of the invention comprises a second coding region which has a nucleic acid sequence that codes for a protein or protein fragment according to the invention, and wherein the first coding region and the second coding region code for non-identical proteins or protein fragments according to the invention.

In a further embodiment, the nucleic acid construct of the invention can comprise one or more further elements for recombinant expression or control of the expression strength or time of the protein or protein fragment.

In a third aspect, the invention relates to a vector system for producing a functional NRPS or PKS, or an NRPS-PKS hybrid, wherein the vector system comprises at least one nucleic acid construct according to the second aspect, and wherein the at least one nucleic acid construct is suitable for expressing at least two proteins or protein fragments according to the first aspect, and wherein the at least two proteins or protein fragments are different and together form a functional NRPS, PKS or an NRPS/PKS hybrid. Thus, the at least two proteins or protein fragments can be expressed via one or two nucleic acid constructs, or, insofar as they are three or more proteins or protein fragments according to the invention, they are expressed by one, two or three nucleic acid constructs. And so on. The vector system of the invention has to provide sufficient coding regions only in its entirety in order to express the desired number of proteins or protein fragments according to the invention.

A preferred embodiment of the invention's vector system relates to a vector system wherein the at least two proteins or protein fragments which can be expressed via the nucleic acid constructs form a functional NRPS, PKS or NRPS/PKS hybrid via the binding between the first and/or second binding domain. This means that the vector system according to the invention comprises, at least in part, those expressible proteins or protein fragments which have the ability to form a functional NRPS/PKS via the binding domains.

Preferably, a functional NRPS, PKS or NRPS/PKS hybrid can synthesise a linear peptide, circular peptide, linear polyketide, circular polyketide, linear peptide-polyketide or circular peptide-polyketide.

In a further embodiment, the preference is for the vector system to comprise nucleic acid constructs which are suitable for the expression of at least three or more proteins or protein fragments according to the invention or wherein at least two of the three or more proteins or protein fragments together form a functional NRPS, PKS or an NRPS/PKS hybrid. Furthermore, the at least three proteins or protein fragments can together form a functional NRPS, PKS or NRPS/PKS hybrid, wherein the functional NRPS or PKS is formed by binding the proteins or protein fragments to one another by means of binding a first binding domain to a second binding domain and binding a third binding domain to a fourth binding domain. In this case, there may be more preference for a first protein or protein fragment to have a terminal first binding domain and an opposite terminal third binding domain, and for a second protein or protein fragment to have a terminal second binding domain, and for a third protein or protein fragment to have a terminal fourth binding domain.

A preferred vector system of the invention is designed in such a way that the binding domains for assembling the NRPS/PKS according to the invention are arranged in such a way that they lie between the NRPS/PKS domains. Preferred arrangements of the binding domains, in particular of the SYNZIPs, can be found in the examples, and specifically only the disclosed position at which the SYNZIPs were incorporated is generalised in an intermediate manner.

In a fourth aspect, the invention relates to a method for producing a functional (complete) NRPS or PKS, or an NRPS/PKS hybrid, comprising bringing at least a first protein or protein fragment according to the first aspect into contact with a second protein or protein fragment according to the first aspect, wherein the first protein or protein fragment has a terminal first binding domain, and wherein the second protein or protein fragment has the terminal second binding domain instead of the terminal first binding domain. As a further step, the method can comprise the recombinant expression of the first and/or second protein or protein fragment by means of at least one nucleic acid construct of the invention.

The terms “the [present] invention”, “according to the invention”, and similar as used here are intended to refer to all aspects, elements, and embodiments of the described and/or claimed invention.

As used in the present disclosure, the term “comprising” is intended to be interpreted to include both “including” and “consisting of”, wherein both meanings are specifically intended, and therefore represent individually disclosed embodiments according to the present invention. The term “and/or is understood as a specific disclosure of each of the two features or components indicated, with or without the other. For example, “A and/or B” is to be understood as a specific disclosure of each of (i) A, (ii) B, and (iii) A and B, as if each were individually disclosed here. In the context of the present invention, the terms “roughly” and “approximately” denote an accuracy interval that experts should understand within a framework in order to still ensure the technical effect of the feature in question. Where an indefinite or specific article is used when referring to a single noun, such as “one” or “the”, such use includes a plural of that noun, unless expressly stated otherwise.

It goes without saying that the application of the teachings of the present invention may be applied to a specific problem or environment and that the inclusion of variants of the present invention or additional features (such as further aspects and embodiments) lies within the capabilities of average experts and in light of teachings contained herein.

Unless otherwise required by context, the descriptions and definitions of the features presented above are not limited to any particular aspect or embodiment of the invention and apply equally to all aspects and embodiments described.

All references, patents and publications cited herein are hereby referred to in their entirety.

In view of the above, it should be noted that the present invention also relates to the following detailed and numbered subject matter:

Subject matter 1: A protein or a protein fragment comprising at least a first domain or partial domain of a non-ribosomal peptide synthetase (NRPS), a polyketide synthase (PKS) or an NRPS/PKS hybrid synth(et)ase (first PKS-NRPS domain), wherein the protein or the protein fragment has an N-terminus or a C-terminus comprising a first binding domain and wherein this first binding domain preferably represents the N-terminus or C-terminus, respectively, of the protein or the protein fragment, and wherein the first binding domain is characterised by the property of being able to enter into a specific protein-protein binding with at least one corresponding second binding domain.
Subject matter 2: The protein or protein fragment of subject matter 1, further comprising at least one, preferably two, three or four or more, further PKS-NRPS domain(s), wherein the further PKS-NRPS domain(s) is/are arranged in a direct functional arrangement next to the first PKS-NRPS domain.
Subject matter 3: The protein or protein fragment of subject matters 1 or 2, wherein the protein or protein fragment does not comprise the at least one corresponding second binding domain.
Subject matter 4: The protein or protein fragment of any one of subject matters 1 to 3, wherein the first binding domain is arranged at the terminal end in such a way that a specific or non-specific mediated interaction/binding to a corresponding second binding domain is possible under normal conditions.
Subject matter 5: The protein or protein fragment of any one of subject matters 1 to 4, wherein the first PKS-NRPS domain, or partial domain, is selected from an A domain, an A-MT domain, a C domain, a C/E domain, an E domain, a Cₛₜₐᵣₜ domain, an FT domain, or a T domain.
Subject matter 6: The protein or protein fragment of any one of subject matters 1 to 5, comprising at least one A or A-MT domain, a C domain and/or E domain or a C/E domain or a Cy domain, and a T domain, wherein the protein or protein fragment preferably has at least one NRPS-PKS elongation module.
Subject matter 7: The protein or protein fragment of any one of subject matters 1 to 6, wherein the binding domain is a protein sequence, preferably a protein domain which mediates a specific protein-protein binding.
Subject matter 8: The protein or protein fragment of any one of subject matters 1 to 7, wherein the binding domain comprises a coiled coil domain.
Subject matter 9: The protein or protein fragment of subject matter 8, wherein the binding domain comprises a synthetic coiled coil domain (SYNZIP).
Subject matter 10: The protein or protein fragment of subject matter 9, wherein the SYNZIP is selected from a SYNZIP 1-23, preferably from SYNZIP 1, 2, 17, 18, or 19. The protein or protein fragment comprising a third binding domain opposite the first binding domain, wherein the third binding domain is characterised by the property of being able to enter into a specific protein-protein binding with at least one corresponding fourth binding domain.
Subject matter 11: The protein or protein fragment of subject matter 11, wherein the first and second binding domains are incapable of binding to the third and fourth binding domains.
Subject matter 12: The protein or protein fragment of subject matter 11, wherein the first and second binding domains may specifically bind to the third or fourth binding domains to form mixtures of peptides.
Subject matter 13: The protein or protein fragment of any one of subject matters 1 to 12, wherein the first binding domain is linked to the first PKS-NRPS domain by a linker sequence.
Subject matter 14: The protein or protein fragment of subject matter 12 or 13, wherein the third binding domain is coupled to the protein or protein fragment via a linker sequence.
Subject matter 15: The protein or protein fragment of one of the preceding subject matters, wherein the binding of the first binding domain to the second binding domain is a non-covalent binding.
Subject matter 16: An isolated nucleic acid construct comprising a first coding region having a nucleic acid sequence encoding a protein or protein fragment of any one of subject matters 1 to 15.
Subject matter 17: The isolated nucleic acid construct of subject matter 16, wherein the first coding region is operatively linked to an expression promoter.
Subject matter 18: The isolated nucleic acid construct of subject matter 16 or 17, further comprising a second coding region having a nucleic acid sequence encoding a protein or protein fragment of any one of subject matters 1 to 15, wherein the first coding region and the second coding region encode non-identical proteins or protein fragments.
Subject matter 19: The isolated nucleic acid construct of any one of subject matters 1 to 18 comprising further elements for recombinant expression of the protein or protein fragment.
Subject matter 20: A vector system for producing a functional NRPS or PKS, wherein the vector system comprises at least one nucleic acid construct according to any one of subject matters 16 to 20, and wherein the at least one nucleic acid construct is suitable for expressing at least two proteins or protein fragments according to any one of subject matters 1 to 15, and wherein the at least two proteins or protein fragments are different and together form a functional NRPS, PKS or NRPS/PKS hybrid.
Subject matter 21: The vector system of subject matter 20, wherein the at least two proteins or protein fragments form the functional NRPS, PKS or NRPS/PKS hybrid via the binding of the first and second binding domains.
Subject matter 22: The vector system of subject claim 20 or 21, wherein a functional NRPS, PKS, or NRPS/PKS hybrid can synthesise a linear peptide, circular peptide, linear polyketide, circular polyketide, linear peptide polyketide, or circular peptide polyketide.
Subject matter 23: The vector system of any one of subject matters 20 to 22, wherein the vector system comprises nucleic acid constructs suitable for the expression of at least three or more proteins or protein fragments according to any one of subject matters 1 to 15, or wherein at least two of the three or more proteins or protein fragments together form a functional NRPS, PKS or an NRPS/PKS hybrid.
Subject matter 24: The vector system of subject matter 23, wherein at least three proteins or protein fragments can together form a functional NRPS, PKS or NRPS/PKS hybrid, wherein the functional NRPS or PKS is formed by binding the proteins or protein fragments to one another by means of binding a first binding domain to a second binding domain and binding a third binding domain to a fourth binding domain.
Subject matter 25: The vector system of subject matters 23 or 24, wherein a first protein or protein fragment has a terminal first binding domain and an opposite terminal third binding domain, and wherein a second protein or protein fragment has a terminal second binding domain, and wherein a third protein or protein fragment has a terminal fourth binding domain.
Subject matter 26: A process for preparing a functional (complete) NRPS or PKS comprising connecting at least a first protein or protein fragment of any one of subject matters 1 to 15 with a second protein or protein fragment of any one of subject matters 1 to 15, wherein the first protein or protein fragment has a terminal first binding domain, and wherein the second protein or protein fragment has the terminal second binding domain instead of the terminal first binding domain.
Subject matter 27: The method of subject matter 26, wherein said connection comprises recombinant expression of said first and/or second protein or protein fragment by means of at least one nucleic acid construct according to any one of subject matters 16 to 19.

### BRIEF DESCRIPTION OF THE FIGURES AND SEQUENCES

The figures show

FIG. 1: shows the SYNZIP interaction partners and possible networks. A) Protein microarray assay results of 26 peptides forming specific interactive pairs. Peptides which are immobilised on the surface of the microarray are shown in series. Fluorescence-labelled peptides in solution are listed in row. According to the array score (shown on the right), black spots show a strong (0-0.2) and white spots a weak fluorescence signal (>1.0). The absence of homospecific interactions is indicated by the red diagonal line. Interactions that showed an array score of <0.2 are highlighted in green. The number of strong interaction partners is shown in the lower column (Reinke et al., 2010). B) Possible SYNZIP interaction networks: 1. linear 2. annular 3. branched and 4. orthogonal networks with the corresponding SYNZIP numbers are indicated. Dashed lines indicate a weak and solid lines a strong interaction. The star highlights the antiparallel interaction between SYZIP17 and SYNZIP18 (Thompson et al., 2012).

FIG. 2: shows the construction of an AmbS hybrid for the production of novel peptides. A: Schematic representation of the NRPS hybrids (NRPS-3a and NRPS-3b) from XUs of the AmbS (black) and GxpS (red). The associated relative peptide production of peptides 7, 8 and 9 from triplicate measurements is shown in %. Symbols represent domains: circle, A domain; rectangle, T domain; triangle, C domain; diamond, C/E domain; small circle at the C-terminus, TE domain. Helices represent SZs: orange, SZ17; green, SZ18. B: Structure of the peptides produced.

FIG. 3: shows the construction of an SzeS hybrid for the production of novel peptides. A: Schematic representation of the NRPS hybrids (NRPS-4a and NRPS-4b) and the covalently linked hybrid (NRPS-4c) from XUs of the SzeS (green) and GxpS (red). The associated relative peptide production of peptides 10 and 11 from triplicate measurements is shown in %. Symbols represent domains: circle, A domain; rectangle, T domain; triangle, C domain or FT domain; diamond, C/E domain; small circle at the C-terminus, TE domain. Helices represent SZs: orange, SZ17; green, SZ18. B: Structure of the peptides produced.

FIG. 4: shows the construction of an XldS hybrid for the production of novel peptides. A: Schematic representation of the NRPS hybrids (NRPS-5a and NRPS-5b) from XUs of the XldS (turquoise) and GxpS (red). The associated relative peptide production of peptides 12, 13, 14 and 15 from triplicate measurements is shown in %. Symbols represent domains: circle, A domain; rectangle, T domain; triangle, C domain; diamond, C/E domain; small circle at the C-terminus, TE domain. Helices represent SZs: orange, SZ17; green, SZ18. B: Structure of the peptides produced.

FIG. 5: shows the proof of concept of various interfaces and SZ oligomerisation status based on the XtpS. Schematic representation of the XtpS (light green) divided in the T-C (NRPS-13), A-T (NRPS-14) and C-A (NRPS-15 and NRPS-16) as well as constructs with different SZ oligomerisation status (NRPS-15 and NRPS-16). The WT-XtpS (NRPS-1) was used as a reference. The relative production of peptides 1 and 2 from triplicate measurements is given in % of the WT level. Symbols represent domains: circle, A domain; rectangle, T domain; triangle, C domain; diamond, C/E domain; small circle at the C-terminus, TE domain. Helices represent SZs: orange, SZ17; green, SZ18, yellow: SZ19.

FIG. 6: shows the influence of the SZs on the production of the A-T divided XtpS. Schematic representation of the three control experiments without N-terminal SZ (NRPS-14b), C-terminal SZ (NRPS-14c) and both SZs (NRPS-14d), as well as representation of the construct with both SZs (NRPS-14a). The WT-XtpS (NRPS-1) was used as a reference. The relative production of peptides 1 and 2 from triplicate measurements is given in % of the WT level. Symbols represent domains: circle, A domain; rectangle, T domain; triangle, C domain; diamond, C/E domain; small circle at the C-terminus, TE domain. Helices represent SZs: orange, SZ17; green, SZ18.

FIG. 7: shows the influence of the SZs on the production of the C-A (SZ19/18) divided XtpS. Schematic representation of the three control experiments without N-terminal SZ (NRPS-16b), C-terminal SZ (NRPS-16c) and both SZs (NRPS-16d), as well as representation of the construct with both SZs (NRPS-16a). The WT-XtpS (NRPS-1) was used as a reference. The relative production of peptides 1 and 2 from triplicate measurements is given in % of the WT level. Symbols represent domains: circle, A domain; rectangle, T domain; triangle, C domain; diamond, C/E domain; small circle at the C-terminus, TE domain. Helices represent SZs: yellow, SZ19; green, SZ18.

FIG. 8: shows the influence of GS linkers on the production of the C-A (SZ17/18) divided XtpS. Schematic representation of the construct without GS linkers (NRPS-15a) and with a to AS long (NRPS-15b), 8 AS long (NRPS-15c) and 4 AS long GS linker (NRPS-15d), which was introduced between the C-terminal end of the first XtpS section and SZ 17. The WT-XtpS (NRPS-1) was used as a reference. The relative production of peptides 1 and 2 from triplicate measurements is given in % of the WT level. Symbols represent domains: circle, A domain; rectangle, T domain; triangle, C domain; diamond, C/E domain; small circle at the C-terminus, TE domain. Helices represent SZs: orange, SZ17; green, SZ18.

FIG. 9: shows the productivity of the three-part XtpS. Schematic diagram of the XtpS divided into the T-C(NRPS-17a) and A-T (NRPS-18a) linkers and corresponding negative control (NRPS-18b). The WT-XtpS (NRPS-1) was used as a reference. The relative production of peptides 1 and 2 from triplicate measurements is given in % of the WT level. Symbols represent domains: circle, A domain; rectangle, T domain; triangle, C domain; diamond, C/E domain; small circle at the C-terminus, TE domain. Helices represent SZs: orange, SZ17; green, SZ18; dark blue: SZ1; light blue: SZ2.

FIG. 10: shows the productivity of the three-part GxpS. A: Schematic representation of the GxpS (NRPS-20) shared in the A-T linkers. The WT-GxpS (NRPS-2) was used as a reference. The relative production of peptides 3, 4, 5 and 6 from triplicate measurements is given in % of the WT level. Symbols represent domains: circle, A domain; rectangle, T domain; triangle, C domain; diamond, C/E domain; small circle at the C-terminus, TE domain. Helices represent SZs: orange, SZ17; green, SZ18; dark blue: SZ1; light blue: SZ2. B: Structure of the peptides produced.

FIG. 11 shows the reprogramming of the XtpS for the production of novel peptides. A: Schematic representation of the hybrids NRPS-23b and NRPS-23c, which were produced by substituting the XtpS (light green) tridomain with a GxpS (red) and SzeS (green) tridomain. The relative production of peptides 16a/b, 17a, 18 and 19a/b from triplicate measurements is shown in % as normalised in comparison to WT (NRPS-1). The tripartite division of XtpS (NRPS-18) is also shown. Symbols represent domains: circle, A domain; rectangle, T domain; triangle, C domain; diamond, C/E domain; small circle at the C-terminus, TE domain. Helices represent SZs: orange, SZ17; green, SZ18; dark blue: SZ1; light blue: SZ2. B: Structure of the peptides produced.

FIG. 12 shows the reprogramming of the XtpS for the production of novel peptides. A: Schematic representation of the hybrids NRPS-24a and NRPS-24c, which were produced by the substitution of the GxpS (red) tridomain with an XtpS, (light green) and SzeS (green) tridomain. The relative production of peptides 20, 21a/b, 22, 23a/b, 24, 25, 3 and 2 from triplicate measurements is shown in % in comparison with WT (NRPS-2). The tripartite division of GxpS (NRPS-20) is also shown. Symbols represent domains: circle, A domain; rectangle, T domain; triangle, C domain; diamond, C/E domain; small circle at the C-terminus, TE domain. Helices represent SZs: orange, SZ17; green, SZ18; dark blue: SZ1; light blue: SZ2 B: structure of the peptides produced.

FIG. 13: shows the design of XtpS hybrids for the production of novel peptides. A: Schematic representation of the hybrids NRPS-26 and NRPS-27, which were each produced from parts of the GxpS (red) and SzeS (green) as well as XtpS (light green). The associated relative peptide production of peptides 20, 22 and 26 from triplicate measurements is shown in %. The tripartite division of XtpS (NRPS-18) is also shown. Symbols represent domains: circle, A domain; rectangle, T domain; triangle, C domain or FT domain; diamond, C/E domain; small circle at the C-terminus, TE domain. Helices represent SZs: orange, SZ17; green, SZ18; dark blue: SZ1; light blue: SZ2. B: Structure of the peptides produced.

FIG. 14: shows the design of GxpS hybrids for the production of novel peptides. A: Schematic representation of the hybrids NRPS-28 and NRPS-29, which were each produced from parts of the XtpS (light green) and SzeS (green) and GxpS (red). The associated relative peptide production of peptides 16a/b, 27, 17a/b, 28a/b, 18, 19, 29, 30, 31a/b and 32 from triplicate measurements is shown in %. The tripartite division of GxpS (NRPS-20) is also shown. Symbols represent domains: circle, A domain; rectangle, T domain; triangle, C domain or FT domain; diamond, C/E domain; small circle at the C-terminus, TE domain. Helices represent SZs: orange, SZ17; green, SZ18; dark blue: SZ1; light blue: SZ2. B: Structure of the peptides produced.

FIG. 15: shows the division of a hybrid of NRPS and PKS modules. The substance produced by the hybrid is glidobactin A (see structure). The NRPS GlbD was shared between the A and T domains with SZs. Symbols represent domains: circle, A domain; rectangle, T domain; triangle, C domain; PKS domains in GlbB are named according to their functions. Helices represent SZs: light grey, SZ17; dark grey, SZ18.

FIG. 16 shows a preferred embodiment in which the sequence of the Synzpi variants SZ1 and SZ2 (A) or SZ2 and SZ19 (B) were shortened in each case at the N terminus. The shortened but still fully functional syncip results in improved peptide production within the NRPS.

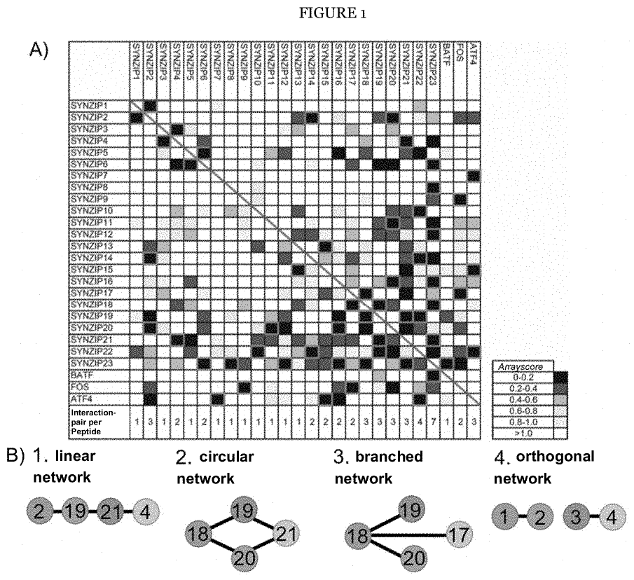

The sequences show:

SEQ ID Nos 1 and 2: synzip sequences

SEQ ID Nos 3 and 5: preferred sequence motifs for inserting the binding domains according to the invention

SEQ ID Nos 6 to 30: peptide sequences of the NRPS peptides produced in this application

### EXAMPLES

Certain aspects and embodiments of the invention will now be illustrated by way of example and with reference to the descriptions, figures and tables set forth herein. Such examples of the substances, processes, uses and other aspects of the present invention are only representative and should not be understood as limiting.

The examples show:

### Example 1: SYNZIP Mediated De Novo Design of NRPSs Based on the XU Concept

This work initially dealt with the de novo construction of NRPSs and the production of novel peptides based on the XU concept. By introducing the SYNZIP pair 17/18 into the conserved WNATE motif of the C-A linker, hybrid NRPs were to be constructed from two systems. The antiparallel SZ pair should serve as a non-covalent mediator between the various synthetases. With a dissociation constant (Kd) of <10 nM, SZ17 and SZ18 have a strong affinity to one another (Thompson et al., 2017), so that almost all properties of a covalent linkage are present. The NRPS hybrids were to be generated by combining the first two XUs of the AmbS, SzeS and XldS with the last three XUs of the GxpS SZ17 was added to the C-terminal end of the AmbS, SzeS and XldS and SZ18 to the N-terminal end of the GxpS. With regard to the rule established by Bozhüyük et al., which requires the consideration of C-domain specificity, the specificities for the first two hybrids (AmbS-GxpS and Sze-GxpS) were observed, but not for the last one (XldS-GxpS).

### Example 2 Plasmid Construction and Heterologous Expression of GxpS Hybrids in E. coli DH10B::mtaA

The first two XUs of AmbS (A1-C3), SzeS (C1-C3) and XldS (C1-C3) were first amplified using the gDNA from X. miraniensis DSM 17902, X. szentirmaii DSM 16338 and X. indica DSM17382. For this purpose, the primers listed in Table 3 were used. These contained matching overhangs to a pACYC_ara_araE vector which already contained the sequence of SZ17. After linearisation of the vector, the plasmids pJW91 (ambS_A1-C3_SZ17), pJW92 (szeS_C1-C3_SZ17), and pJW93 (xldS_C1-C3_SZ17) were cloned from plasmid backbone and inserts by a hot fusion reaction (see 2.3.7). After screening and verification of the plasmids, they were each transformed together with a further plasmid pJW76 (SZ18_gxpS_A3-TE) or pJW83 (gxpS_A3-TE) into E. coli DH10B::mtaA. In doing so, pJW76 contained the sequence of the last three XUs of the GxpS and the sequence of the SZ18. In contrast, the transformation of pJW83, which lacks the sequence of SZ18, served as a negative control. Protein production was carried out by induction with L(+)-arabinose in triplicates at 22° C. for 72 hours.

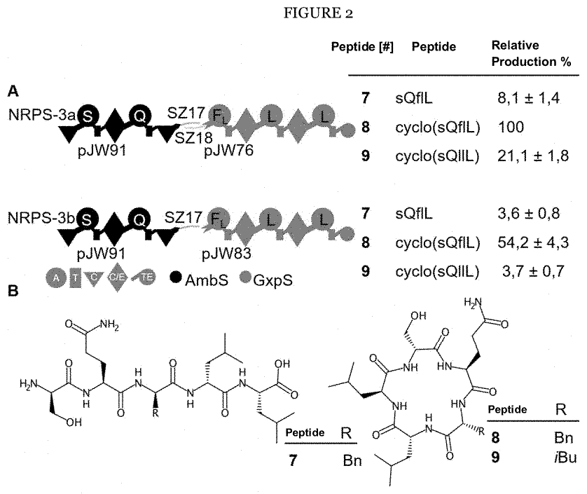

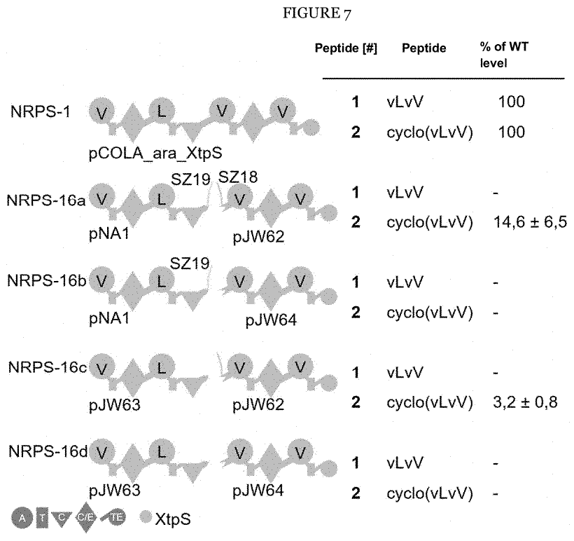

In the subsequent analysis by means of HPLC-MS (see 2.5.2), a search was carried out for the masses of the peptides which would result from the hybrid systems. Accordingly, m/z values of 607.23 [M+H⁺] for 7 (linear peptide) and 589.33 [M+H⁺] 8 (cyclic peptide) were sought for the hybrid NRPS-3a, which was composed of parts of the AmbS and GxpS. These masses could be calculated from the peptide sequence (sQflL). Due to the promiscuity of the third GxpS A domain, which is capable of incorporating leucine in addition to phenylalanine, m/z values of 573.36 [M+H⁺] (linear peptide) and 555.35 [M+H⁺] (9) (cyclic peptide) were also searched for. In this case, the masses resulted from the sequence (sQllL). The peptides 7, 8 and 9, which eluted at a retention time of 6 min, 7.1 min and 7 min, could be identified on the basis of their mass. The linear peptide with the sequence (sQllL), on the other hand, could not be detected. Since no standard was available at the time of data acquisition, the quantification of the results was carried out relatively and was calculated from the mean value of the peak area (FIG. 2). This showed that 8 was the most frequently detected peptide. 7 and 9 were produced at 8.1% and 21.2% relative to 8. All peptides could be verified on the basis of their MS² spectrum (Annex FIG. 2). Furthermore, the measurement data of the negative control (NRPS-3b) showed that the production of 7, 8 and 9 is also possible without N-terminal SZ (FIG. 7). However, the production of the peptides was significantly lower than in the comparison system with both SZs (NRPS-3a). Thus, 7 and 8 were only produced to ˜50% and peptide 9 to ˜18%.

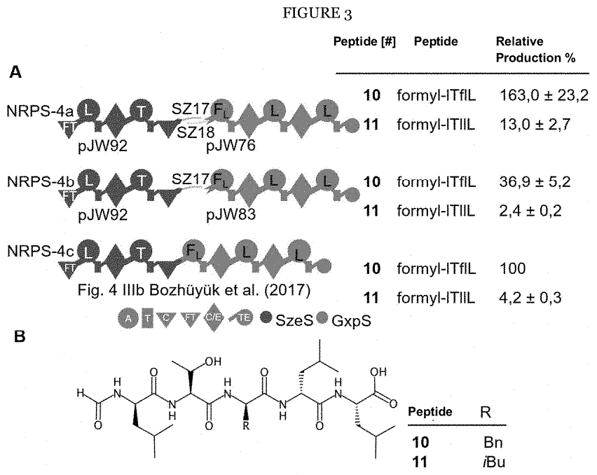

For the second hybrid NRPS-4a, which was composed of the first two XUs of SzeS and the last three XUs of GxpS, data was searched in the recorded HPLC-MS for m/z values of 634.38 [M+H⁺] for the phenylalanine derivative 10 (formyl-1TflL) and 601.36 [M+H⁺] for the leucine derivative 11 (formyl-1TllL). The construct without N-terminal SZ (NRPS-4b) and the covalently linked system (NRPS-4c) served as comparison systems. Both the mass of 10 and the mass of 11 could be detected in the extracted ion chromatogram (EIC) of the measured data, which eluted in each case at a retention time of 8 minutes and 7.8 minutes. Peptide 10 was most frequently determined and peptide 11 was determined in relative values of 8% (FIG. 3). Furthermore, the measurement data of the negative control (NRPS-4b) showed a significant decrease of 10 and 11 by ˜80%. The covalently linked construct (NRPS-4c) also produced the peptides in significantly smaller amounts. Thus, only ˜60% of 10 and ˜30% of ii were detected relative to NRPS-4a.

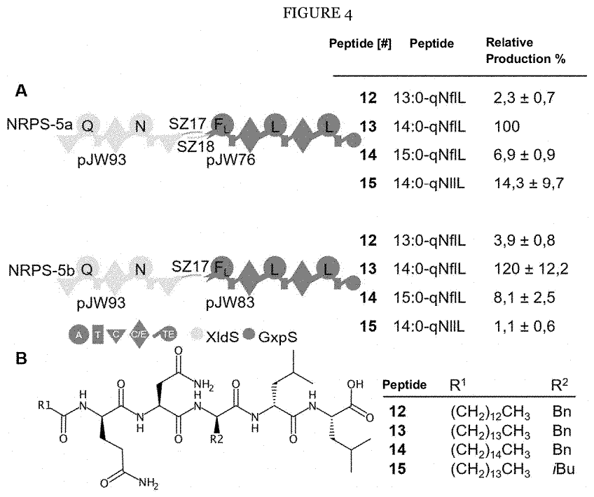

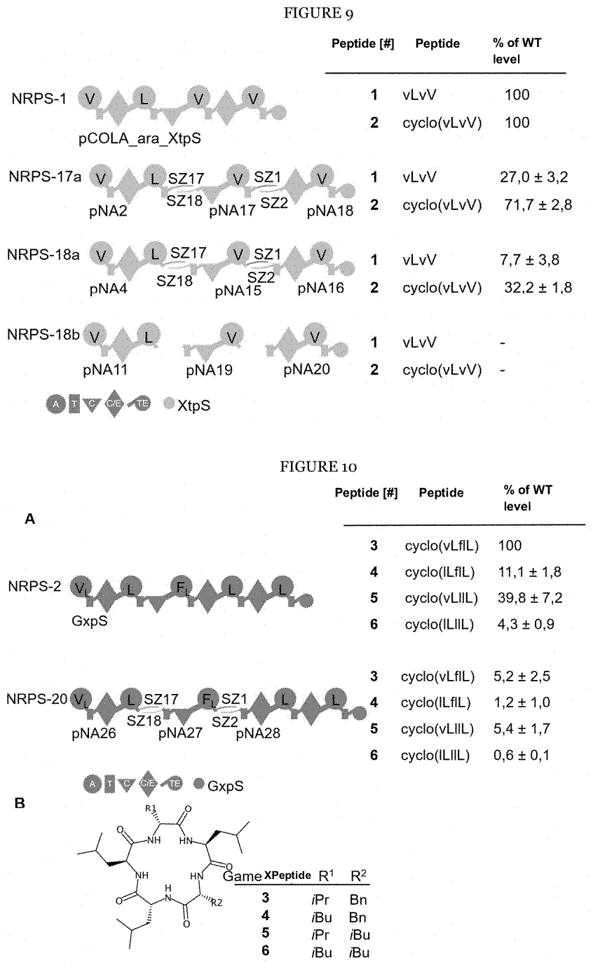

Ultimately, the HPLC-MS data of the third hybrid (NRPS-5a), which was composed of parts of the XldS and GxpS, showed the production of most expected derivatives (FIG. 4). Since the C1 domain of the XldS permits the incorporation of a C13, C14 or C15 FS at the N-terminal end of the peptide, the promiscuity of the third GxpS A domain results in six possible derivatives with m/z values of 830.54 [M+H⁺] 12 (13:0-qNflL), 844.55 [M+H⁺] 13 (14:0-qNflL), 858.12 [M+H⁺] (15:0-qNllL), 796.55 [M+H⁺] (13:0-qNllL), 810.57 [M+H⁺] 15 (14:0-qNllL) and 824.59 [M+H⁺] (15:0-qNllL). Four of them were detected. The retention times of the C13, C14 or C15 derivatives were in each case 11.3 min, 11.8 min and 12.3 min. While 13 was the most frequently produced peptide, the remaining peptides were detected in relative amounts between 2.3% (12) and 14.3% (15) (FIG. 9). Furthermore, the signal intensity of the EICs was low, indicating low overall production. In addition, the negative control (NRPS-5b) showed no significant difference in peptide production from the NRPS-5a construct (FIG. 4).

### Example 3: Strategies for the SYNZIP-Mediated Reconstruction of NRPSs

Above, the conserved WNATE motif (SEQ ID No 3) of the C-A linker was chosen as the preferred cleavage site on the basis of the XU concept. This cleavage site was postulated by Bozhüyük et al. from sequence alignments of NRPS linker regions from Photorhabdus and Xenorhabdus and from published NRPS structural data of other organisms as an ideal fusion point. Also mentioned was that the A-T and T-C linker regions are less suitable for the reprogramming of NRPS because of their low conserved sequence compared with the C-A linker. With the introduction of the SZ pair 17/18 into the T-C and T-A linker regions, a comparative test should nevertheless be carried out to determine whether these insertion sites are not also equally suitable fusion points. This hypothesis was checked using the XtpS model system. To this end, XtpS was aligned with the structural data of bacillibactin synthetase from Bacillus subtilis (Tarry et al., 2017), which was published in 2017, in order to obtain conclusions about possible secondary structures. Subsequently, on the basis of this, cleavage sites in the T-C and A-T linker region were defined, which ultimately related to the sequence motifs RV|LP (SEQ ID No 4) of the T-C linker and VY|AAP (SEQ ID No 5; vertical line illustrates cleavage site) of the A-T linker.

Furthermore, two different SYNZIP oligomerisation statuses should be compared, which mean that the XtpS subunits bound to the SZs are either in spatial proximity or further apart. In principle, both orientations can be implemented with both parallel and antiparallel SZ. However, only the conformation in which the proteins are further apart is practicable. The reason for this is that after dividing the NRPS system in two, the SZs can only be attached to two (C-terminus of the first and N-terminus of the second NRPS part), instead of four possible terms (N- and C-terminus of the first and N- and C-terminus of the second protein part) of the proteins. By introducing SZ19 and the functional reverse form of SZ18, however, a close orientation seems possible. Nevertheless, this pair is not characterised by Thompson et al. (only SZ19 with the forward form of SZ18), and accordingly data on the Kd value, interaction partners, etc., is missing. Ultimately, the antiparallel SZ pair 17/18 was used for wide conformation and the parallel SZ pair 19/18 for close conformation.

For the XtpS divided in the T-C and A-T linkers (NRPS-13 and NRPS-14, cleavage site see 3.2), in each case two plasmids, pNA2 (xtpS_A1-T2_SZ17) and pNA3 (SZ18_xtpS_C3-TE), and pNA4 (xtpS_A1-A2_SZ17) and pNA5 (SZ18_xtpS_T2-TE), were assembled and together transformed in E. coli DH10B::mtaA. In contrast, the plasmids pJW61 (xtpS_A1-C3_SZ17) and pJW62 (SZ18_xtpS_A3-TE) were used to represent the cleavage site in the C-A linker. Furthermore, for the NRPS-16 divided in the C-A linker, the C-terminal SZ17 was replaced by SZ19, while the N-terminal SZ18reverse remained unchanged. The production by the wild-type XtpS (NRPS-1) served as a reference. Production cultures of all constructs were simultaneously prepared as triplicates and the synthesis of 1 and 2 was tested by means of HPLC-MS.

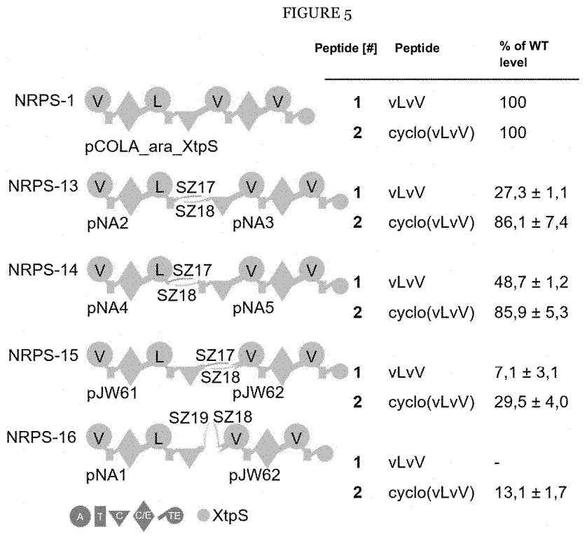

Since an absolute quantification was not possible due to a missing standard, a relative evaluation of the peak area was carried out (FIG. 5). Different from the relative values in FIG. 5, the linear peptide 1 is not formed in virtually the same amounts as the cyclic peptide 2, based on the absolute peptide yield, but only at about 0.1%. This result comes about merely because of the better ionisation of the linear peptide and must be taken into account when considering the relative values. The measurement results showed that the production of the cyclic product 2, with an m/z value of 411.31 [M+H+], and in most cases also the production of the linear peptide 1, with an m/z value of 429.31 [M+H+], could be demonstrated for all constructs (FIG. 5). 2 was best produced with about 80% relative to the WT by the constructs divided in the T-C(NRPS-13) and A-T (NRPS-14) linkers, whereas the two constructs divided in the C-A, NRPS-15 and NRPS-16, showed a significantly lower production with 27% (NRPS-15) and 13% (NRPS-16). The linear peptide (1) was produced in negligibly smaller amounts better by NRPS-14 instead of NRPS-13 and NRPS-16 showed no production of 1.

### Example 5: Influence of the SYNZIPs on the Production of the A-T Divided XtpS

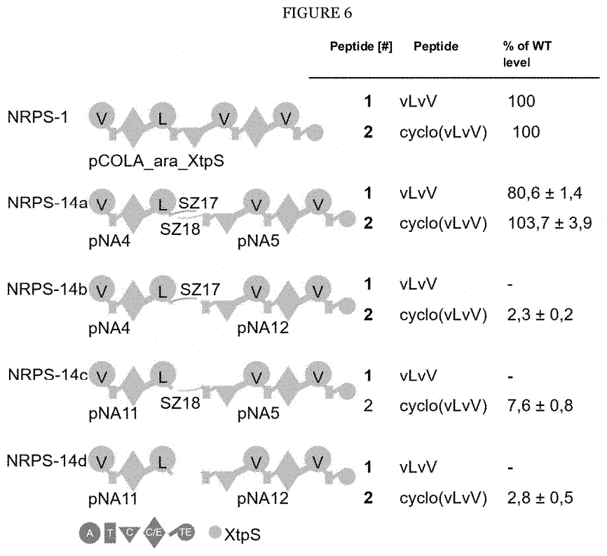

The influence of the SZs on the production of 1 and 2 was examined for the A-T divided construct NRPS-14 a (FIG. 6). After assembling pNA11 (xtpS_A1-A2) and pNA12 (xtpS_T2-TE), control experiments were carried out in which, in the first case, the N-terminal (NRPS-14b), in the second case the C-terminal (NRPS-14c) and in the third case both SZs (NRPS-14c) (FIG. 6) were left out. For this purpose, the plasmids pNA12 (xtpS_T2-TE) and pNA11 (XtpS_A1-A2) were cloned, each of which lacked the sequence of SZ17 and SZ18. The results of the HPLC-MS data are shown in FIG. 6. The negative controls of the A-T divided construct (NRPS-14b, NRPS-14c and NRPS-14d) showed a very low production of the cyclic product 2 and absolutely no production of the linear peptide 1 at ˜3-10% of the WT level. Overall, the controls showed a decrease in productivity of 90% compared to NRPS-14a (FIG. 6). Furthermore, NRPS-14a produced the peptides 2 and 1 with 104% and 81% at WT level.

### Example 6: Influence of the SYNZIPs on the Production of the A-C(5Z19/18) Shared XtpS

The same control experiments were likewise carried out for the A-C divided construct with SZ pair 19/18. Since this construct already showed low production with both SZs (FIG. 5), it was possible to detect only a very low or no production of 2 and 1 for the three control experiments carried out (FIG. 7). The relative analysis of the HPLC-MS measurement data showed no peptide production for control experiments NRPS-16b and NRPS-16d; NRPS-16c showed only a very low production of 2 with 3.2% of the WT level. In relation to the construct with both SZs (NRPS-16a), control NRPS-16c shows a decrease in the production of cyclic peptide 2 by 80%.

### Example 7: Influence of GS Linkers on the Production of A-C(SZ17/18) Shared XtpS

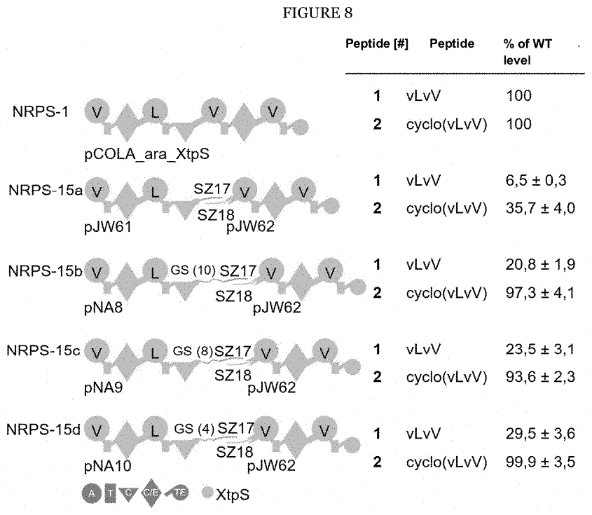

Since the C-A (NRPS-15, FIG. 5), compared to the T-C(NRPS-13, FIG. 5) and A-T (NRPS-14, FIG. 5) divided XtpS construct, showed considerably poorer productivity, GS linkers of different lengths were introduced between the C-terminal end of the first XtpS section and SZ17, with the aim of increasing productivity. Since, according to the XU concept published by Bozhüyük et al. for the construction of reprogrammed NRPS, ten AS of the conserved WNATE motif were deleted and the same happened to the C-A construct (NRPS-15) shown in FIG. 8, the introduction of a ten AS long GS linker was started. This was achieved by assembling the plasmid pNA8 (xtpS_A1-C3_GS(10)_SZ17), a plasmid derived from pJW61 (A1-C3_SZ17). In addition, two further plasmids, pNA9 (xtpS_A1-C3_GS(8)_SZ17) and pNA10 (xtpS_A1-C3_GS(4)_SZ17), were constructed, which code for an eight and four AS long GS linker. The evaluation of the HPLC-MS measurement data showed a better production of all constructs (NRPS-15b, NRPS-15c, NRPS-15d) with GS linker compared to the construct without it. Overall, the introduction of a linker resulted in an average increase in productivity of ˜37% for cyclic 2 and ˜26% for linear peptide 1. Furthermore, the cyclic product 2 with almost WT level was produced for all constructions with GS linker.

### Example 7: Proof of Concept: Productivity of a Three-Part XtpS System

Since the division of the XtpS into two parts was successful for each of the positions mentioned above, the next step was to divide the system into two parts. For this purpose, a further SYNZIP pair, SZ1 and SZ2, was introduced which does not communicate with SZ17 and SZ18 and thus forms a so-called orthogonal network. With a Kd value of <10 nM, SZ1 and SZ2, as well as SZ17 and SZ18, show a very strong affinity for one another. Furthermore, only the A-T and T-C linker regions were selected as positions for the three-part division, which proved to be the most favourable positions with the best production through NRPS-13 and NRPS-14 (FIG. 5). Accordingly, two constructs, NRPS-17a and NRPS-18a, were produced, which were each divided into the linker regions T-C and A-T, respectively, with the introduction of the two SZ pairs. In detail, the SZs were introduced into the second and third T-C linkers or second and third A-T linkers. A total of four further plasmids, namely pNA17 (SZ18_xtpS_C3-T3_SZ1) and pNA18 (SZ2_xtpS_C4-TE), were cloned for the division in the T-C, and pNA15 (SZ18_xtpS_T2-A3_SZ1) and pNA16 (xtpS_SZ2_T3-TE) were cloned for the division in the A-T region. In addition, the plasmids were assembled without SZs as negative controls. This resulted in pNA19 (xtpS_T2-A3) and pNA20 (xtpS_T3-TE) for the negative control of the A-T split (NRPS-18b, FIG. 9).

For both three-part constructs, NRPS-17a and NRPS-18a, the production of the linear 1 and cyclic 2 peptide could be identified (FIG. 9). In comparison to NRPS-18a, NRPS-17a produced the peptides in two (2) to more than three times the amount (1). Accordingly, 2 was identified as 71.7% and 32.2%, respectively, and 1 as 25.6% and 7.3%, respectively. Furthermore, the negative control of the A-T divided system (NRPS-18b) showed no production of the peptides.

### Example 8: SYNZIP-Mediated Tridomain Exchange for the Construction of Hybrid NRPs

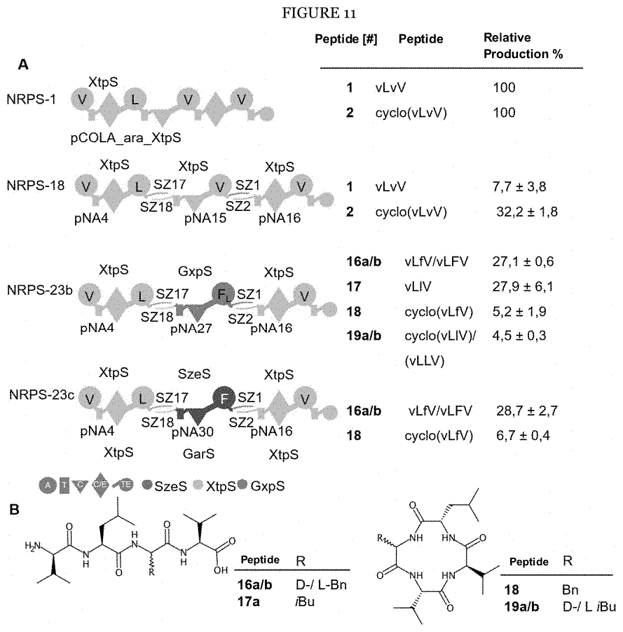

In addition to XtpS, GxpS (NRPS-20) were also divided into three parts (FIG. 10) and tridomain sections of XldS and SzeS were produced. The resulting tridomain sections of the systems should then be combined with one another in a further experiment for the production of novel peptides. From sequence alignments of all A-T and T-C linker regions of the four systems, the cleavage site of the A-T linker (see 3.2, slight variation of the sequence motif within and between the systems) turned out to be a more favourable cleavage site (sequence motif of the cleavage site more conserved). Accordingly, only the interface in the A-T linker was used for all other constructs shown. This means that the substrate specificity of the downstream C domain should no longer be taken into account, as in the XU concept, but that of the upstream C domain. In addition to XtpS, NRPS-23 also consists of parts of the GxpS and SzeS (FIG. 11). After checking the productivity of the three-part NRPS-18, NRPS-23b and NRPS-23c, the tridomains were interchanged.

### Example 9: Productivity of Further Systems Divided into Three Parts

For the construction of the GxpS (NRPS-18) and SzeS (NRPS-19) systems divided into three parts, a set of three plasmids was cloned in each case. This resulted in the plasmids pNA26 (gxpS_A1-A2_SZ17), pNA27 (SZ18_gxpS_T2-A3_SZ1) and pNA28 (SZ2_gxpS_T3-TE) as well as pNA29 (szeS_C1-A2_SZ17), pNA30 (szeS_T2-A3_SZ1) and pNA31 (SZ2_szeS_T3-TE), which were each transformed jointly in E. coli DH10B::mtaA.

For the NRPS-20 (three-part GxpS), all four derivatives 3, 4, 5 and 6 with m/z values of 586.40 [M+H+], 600.41 [M+H+], 552.41 [M+H+] and 566.43 [M+H+] in each case could be determined (FIG. 10). Compared to the WT-NRPS (NRPS-2), however, productivity was greatly reduced. For example, only 5.2% of 3 was produced, only 10.8% of 4 and only ˜14% of 5 and 6 (FIG. 10).

### Example 10: The Exchange of Tridomains for the Production of Novel Peptides

The division of the described NRPSs based on three plasmids makes manipulation of the systems simple. With the experimental implementation, instead of the original plasmids, one or two plasmids are replaced and transformed together in a new constellation into the respective expression strain. The post-translational communication between the various NRPSs is then mediated by the artificial leucine zippers. Thus, for example, the second plasmid of the XtpS set can be replaced by the second plasmid of the GxpS set, thereby constructing a new hybrid system. Overall, the plasmids produced in this work permit the construction of 50 hybrid synthetases. In the following examples, 8 of them are described.

The first tridomain exchange was intended to allow the substitution of the second valine of Xtp for a phenylalanine. In the experimental implementation, instead of the pNA15 plasmid, the plasmids pNA27 and pNA30 were transformed together with pNA4 and pNA16 in E. coli DH10B::mtaA, thereby enabling the production of the hybrids NRPS-23b and NRPS-23c (FIG. 11).

The relative evaluation of the HPLC-MS analysis showed the peptides to be expected for NRPS-23b and NRPS-23c (FIG. 11). Thus, both the phenylalanine derivative and the leucine derivative in linear form (16, 17) and cyclic form (18, 19) were detected by NRPS-23b. In the EIC of peptides 16 and 19, there were also double peaks in each case which had different retention times, but showed identical fragmentation of the MS2 spectrum. From this, it was concluded that the peptides occurred as stereoisomers and eluted accordingly at different times. Since a non-natural protein-protein interface exists exclusively for the last C/E domain of the hybrid NRPS-23b, it was deduced that the upstream AS occurs in two conformations. In the example of 16 and 19, these are in each case the AS phenylalanine and leucine. However, which AS is actually affected has not been studied and therefore remains unresolved. Furthermore, the peptides 16 and 18 were produced by NRPS-23c, as already produced by NRPS-23b. A double peak occurred again for peptide 16, for which reason stereoisomers 16a and 16b were assumed. For the relative evaluation of the isomers, the peak areas were added.

The most frequently detected peptides were the linear peptides 16a/b (in both hybrids) and 17, all of which were produced in virtually identical amounts of 94.4%-100% (FIG. 11). The influence of ionisation on the frequency of the linear peptide is discussed in section 4. Overall, both hybrids, NRPS-23b and NRPS-23c, showed a similar production of the peptides 16a/b and 18, after which 16a/b were produced with 94.4% and 100% respectively and 18 with 19.1% and 24.8% respectively. Furthermore, very similar values could also be determined for the phenylalanine derivatives (16a/b and 18) and leucine derivatives (17a and 19a/b) which were produced by NRPS-23b.

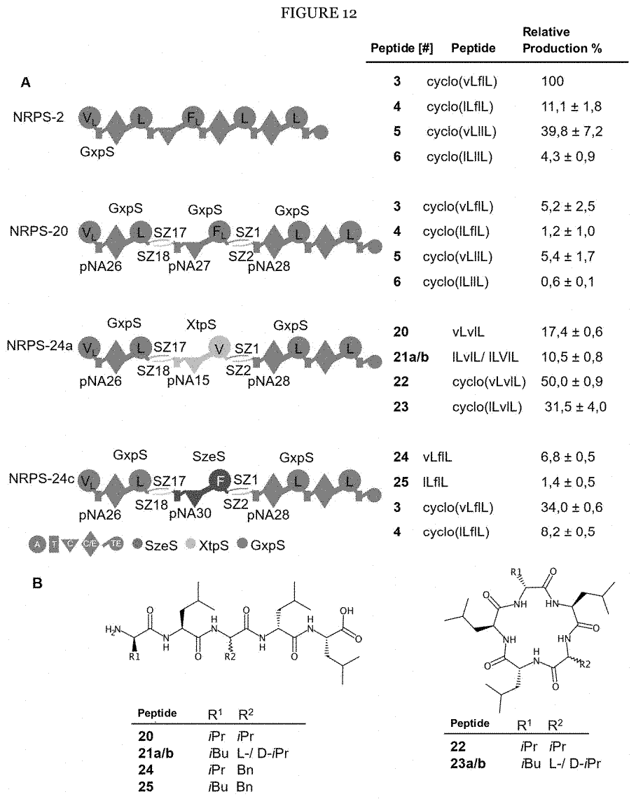

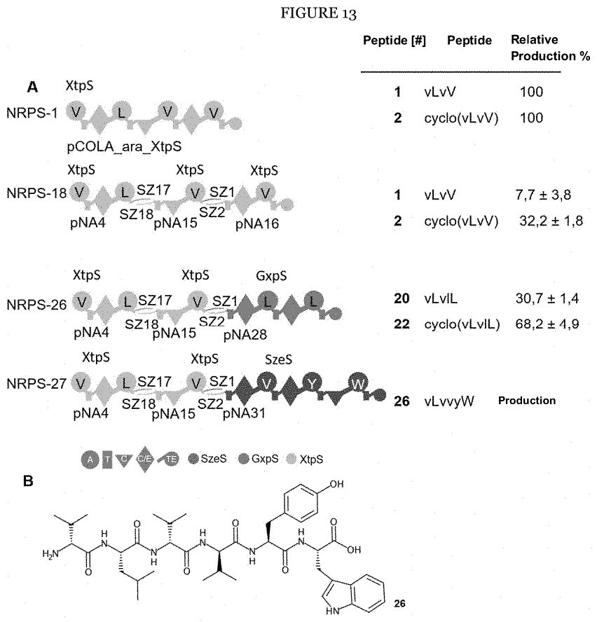

In the second tridomain exchange, the substitution of the phenylalanine of the Gxps by the valine (from Xtp) or phenylalanine (from Sze) should take place. By replacing pNA27 with pNA15 and pNA30 in each case, the hybrids NRPS-24a and NRPS-24c were produced (FIG. 12).

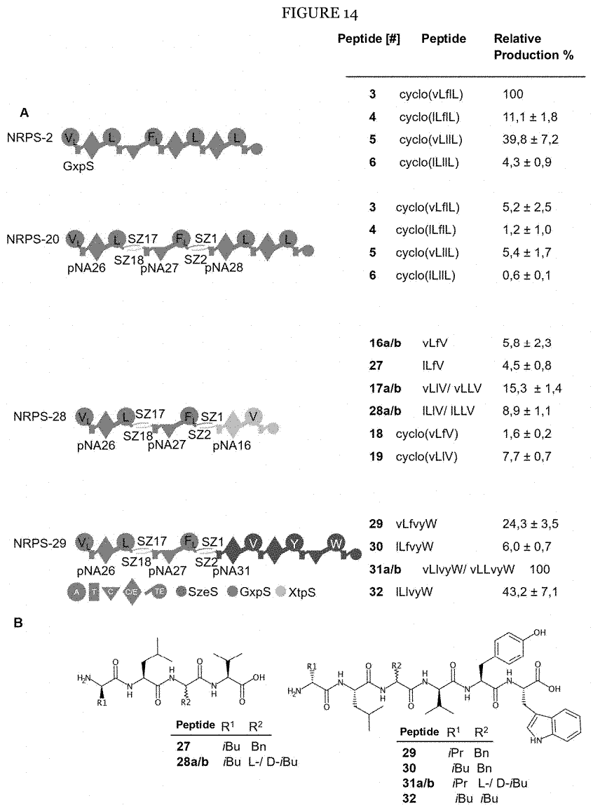

The relative analysis of the measured data showed that peptide production could be determined for NRPS-22a and NRPS-22c. Both hybrids produced both the valine derivative (20, 22, 24 and 3) and the leucine derivative (21a/b, 23, 25 and 4) in linear and cyclic form (FIG. 12). The most commonly produced peptide of the hybrid NRPS-24a was the cyclic valine derivative 22. The linear shape (20) was detected at 34.7% relative to 22. Furthermore, the leucine derivative was produced in cyclic form (23) at 62.9% in smaller amounts than 22%. The linear peptide 21 occurred with relative values of 20.9% and was detected as a stereoisomer (21a and 21b). NRPS-24c showed an overall poorer production compared to NRPS-24a. Thus, the cyclic valine derivative 3 was produced to the extent of 66.1% compared with 22, and the cyclic leucine derivative 4 was detected to the extent of only 16.3% in relative values. Furthermore, the linear peptides 24 and 25 were detected in NRPS-22c at 13.4% and 2.7% in smaller amounts than the cyclic peptides. The structures of the peptides are shown in FIG. 12B. Further novel peptides could correspondingly be obtained by further combinations of the corresponding plasmids and are shown in FIGS. 14 and 14.

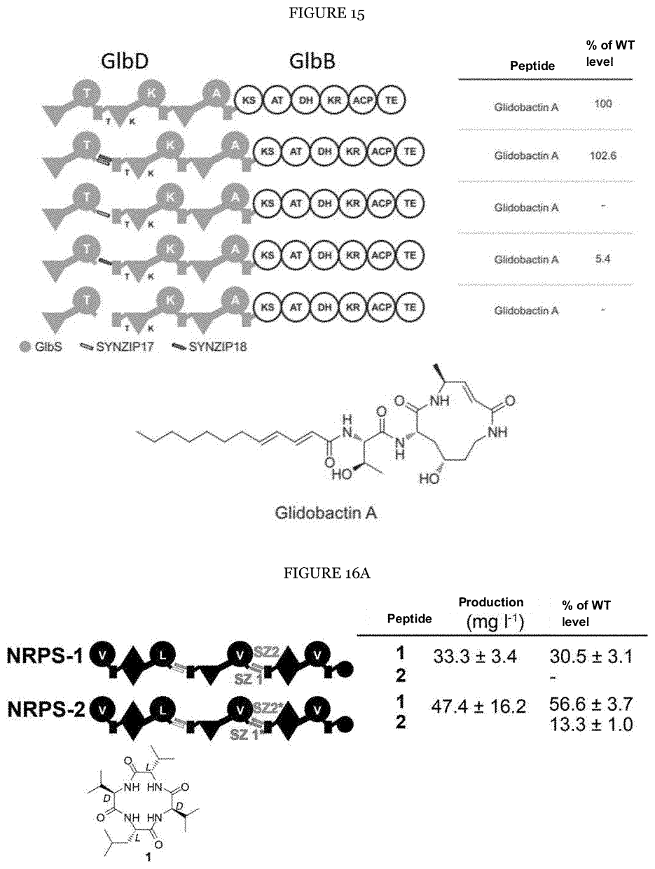

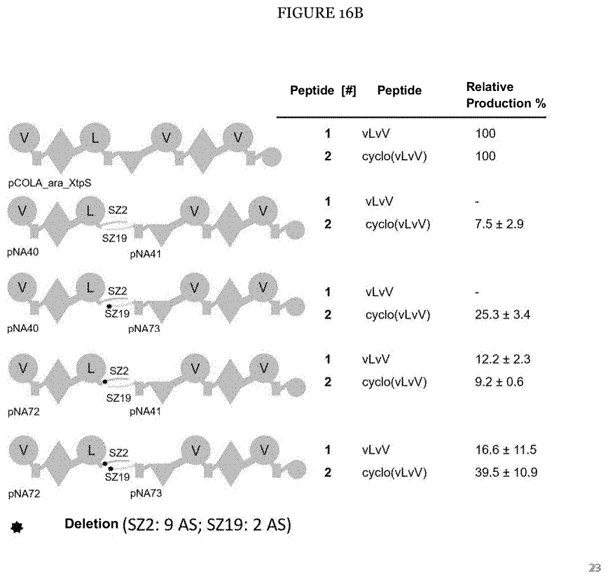

Furthermore, the hybrid of PKS and NRPS modules shown in FIG. 15 was generated, which leads to a complete synthesis of the glidobactin peptide. For this purpose, GlbD was divided between A-T and SZ17/SZ18. In the negative controls without in each case one or both SZs, there is virtually no glidobactin A production.

The following plasmids were used in the context of the examples:

| Name | Genotype | Reference |
|---|---|---|
| pACYC_ara_araE | ori p15A, cm^{R}, araC-P_{BAD}, tacI- | AK Bode |
| | araE, MCS | |
| pCOLA_ara_tacI | ori ColA, kan^{R}, araC-P_{BAD}, tacI, | AK Bode |
| | MCS | |
| pCDF_ara_tacI | ori ColDF13, spek^{R}, araC-P_{BAD} | AK Bode |
| | tacI, MCS | |
| pACYC_ara_XtpS | ori p15A, cm^{R}, araC-P_{BAD} XtpS | Watzel, 2019 |
| | and tacI-araE | (unpublished) |
| pACYC_ara_GxpS | ori p15A, cm^{R}, araC-P_{BAD} GxpS | Watzel, 2019 |
| | and tacI-araE | (unpublished) |
| pA22.3 | ori ColA, kan^{R}, araC-P_{BAD} | Bozhüyük et |
| | szeS_C₁A₁T₁C/ | al., 2018 |
| | E₂A₂T₂C₃_{_{—}}gxpS_A₃T₃C/ | |
| | E₄A₄T₄TE und tacI | |
| pJW61 | ori p15A, cm^{R}, araC-P_{BAD} | Watzel, 2019 |
| | xtpS_A₁T₁C/E₂A₂T₂C₃- | (unpublished) |
| | SYNZIP17 and tacI-araE | |
| pJW62 | ori ColA, kan^{R}, araC-P_{BAD} | Watzel, 2019 |
| | SYNZIP18-xtpS_A₃T₃C/ | (unpublished) |
| | E₄A₄T₄TE and tacI | |
| pJW63 | ori p15A, cm^{R}, araC-P_{BAD} | Watzel, 2019 |
| | xtpS_A₁T₁C/E₂A₂T₂C₃ and | (unpublished) |
| | tacI-araE | |
| pJW64 | ori ColA, kan^{R}, araC-P_{BAD} | Watzel, 2019 |
| | xtpS_A₃T₃C7E₄A₄T₄TE and | (unpublished) |
| | tacI | |
| pJW75 | ori p15A, cm^{R}, araC-P_{BAD} | Watzel, 2019 |
| | gxpS_A₁T₁C/E₂A₂T₂C₃- | (unpublished) |
| | SYNZIP17 and tacI-araE | |
| pJW76 | ori ColA, kan^{R}, araC-P_{BAD} | Watzel, 2019 |
| | SYNZIP18-gxpS_A₃T₃C/ | (unpublished) |
| | E₄A₄T₄TE and tacI | |
| pJW82 | ori p15A, cm^{R}, araC-P_{BAD} | Watzel, 2019 |
| | gxpS_A₁T₁C/E₂A₂T₂C₃ and | (unpublished) |
| | tacI-araE | |
| pJW83 | ori ColA, kan^{R}, araC-P_{BAD} | Watzel, 2019 |
| | gxpS_A₃T₃C/E₄A₄T₄TE and | (unpublished) |
| | tacI | |
| pJW91 | ori p15A, cm^{R}, araC-P_{BAD} | This study |
| | ambS_A₁T₁C/E₂A₂T₂C₃- | |
| | SYNZIP17 and tacI-araE | |
| pJW92 | ori p15A, cm^{R}, araC-P_{BAD} | This study |
| | szeS_C₁A₁T₁C/E₂A₂T₂C₃- | |
| | SYNZIP17 and tacI-araE | |
| pJW93 | ori p15A, cm^{R}, araC-P_{BAD} | This study |
| | xldS_C₁A₁T₁C/E₂A₂T₂C₃- | |
| | SYNZIP17 and tacI-araE | |
| pNA1 | ori p15A, cm^{R}, araC-P_{BAD} | This study |
| | xtpS_A₁T₁C/E₂A₂T₂C₃- | |
| | SYNZIP19 and tacI-araE | |
| pNA2 | ori p15A, cm^{R}, araC-P_{BAD} | This study |
| | xtpS_A₁T₁C/E₂A₂T₂-SYNZIP17 | |
| | and tacI-araE | |
| pNA3 | ori ColA, kan^{R}, araC-P_{BAD} | This study |
| | SYNZIP18-xtpS_C₃A₃T₃C/ | |
| | E₄A₄T₄TE and tacI | |
| pNA4 | ori p15A, cm^{R}, araC-P_{BAD} | This study |
| | xtpS_A₁T₁C/E₂A₂-SYNZIP17 | |
| | and tacI-araE | |
| pNA5 | ori ColA, kan^{R}, araC-P_{BAD} | This study |
| | SYNZIP18-xtpS_T₂C₃A₃T₃C/ | |
| | E₄A₄T₄TE and tacI | |
| pNA6 | ori p15A, cm^{R}, araC-P_{BAD} | This study |
| | xtpS_A₁T₁C/E₂A₂T₂ and tacI- | |
| | araE | |
| pNA7 | ori ColA, kan^{R}, araC-P_{BAD} | This study |
| | xtpS_C₃A₃T₃C/E₄A₄T₄TE and | |
| | tacI | |
| pNA8 | ori p15A, cm^{R}, araC-P_{BAD} | This study |
| | xtpS_A₁T₁C/E₂A₂T₂C₃-GS(10)- | |
| | SYNZIP17 and tacI-araE | |
| pNA9 | ori p15A, cm^{R}, araC-P_{BAD} | This study |
| | xtpS_A₁T₁C/E₂A₂T₂C₃-GS(8)- | |
| | SYNZIP17 and tacI-araE | |
| pNA10 | ori p15A, cm^{R}, araC-P_{BAD} | This study |
| | xtpS_A₁T₁C/E₂A₂T₂C₃-GS(4)- | |
| | SYNZIP17 and tacI-araE | |
| pNA11 | ori p15A, cm^{R}, araC-P_{BAD} | This study |
| | xtpS_A₁T₁C/E₂A₂ and tacI- | |
| | araE | |
| pNA12 | ori ColA, kan^{R}, araC-P_{BAD} | This study |
| | xtpS_T₁C₃A₃T₃C/E₄A₄T₄TE | |
| | and tacI | |
| pNA14 | ori p15A, cm^{R}, araC-P_{BAD} | This study |
| | xtpS_A₁T₁C/ | |
| | E₂A₂T₁C₃A₃T₃C/E₄A₄T₄- | |
| | gxpS_TE and tacI-araE | |
| pNA15 | ori ColA, kan^{R}, araC-P_{BAD} | This study |
| | SYNZIP18-xtpS_T₂C₃A₃- | |
| | SYNZIP1 and tacI | |
| pNA16 | ori CloDF13, spec^{R}, araC-P_{BAD} | This study |
| | SYNZIP2-xtpS_T₃C/E₄A₄T₄TE | |
| | and tacI | |
| pNA17 | ori ColA, kan^{R}, araC-P_{BAD} | This study |
| | SYNZIP18-xtpS_C₃A₃T₄- | |
| | SYNZIP1 and tacI | |
| pNA18 | ori CloDF13, spec^{R}, araC-P_{BAD} | This study |
| | SYNZIP2-xtpS_C/E₄A₄T₄TE | |
| | and tacI | |
| pNA19 | ori ColA, kan^{R}, araC-P_{BAD} | This study |
| | xtpS_T₂C₃A₃ and tacI | |
| pNA20 | ori CloDF13, spec^{R}, araC-P_{BAD} | This study |
| | xtpS_T₃C/E₄A₄T₄TE and tacI | |
| pNA21 | ori ColA, kan^{R}, araC-P_{BAD} | This study |
| | xtpS_C₃A₃T₄ and tacI | |
| pNA22 | ori CloDF13, spec^{R}, araC-P_{BAD} | This study |
| | xtpS_C/E₄A₄T₄TE and tacI | |
| pNA26 | ori p15A, cm^{R}, araC-P_{BAD} | This study |
| | gxpS_A₁T₁C/E₂A₂-SANZIP17 | |
| | and tacI-araE | |
| pNA27 | ori ColA, kan^{R}, araC-P_{BAD} | This study |
| | SYNZIP18-gxpS_T₂C₃A₃- | |
| | SYNZIP1 and tacI | |
| pNA28 | ori CloDF13, spec^{R}, araC-P_{BAD} | This study |
| | SYNZIP2-gxpS_T₃C/E₄A₄T₄ | |
| | C/E₅A₅T₅TE and tacI | |
| pNA29 | ori p15A, cm^{R}, araC-P_{BAD} | This study |
| | szeS_C₁A₁T₁C/E₂A₂-SYNZIP17 | |
| | and tacI-araE | |
| pNA30 | ori ColA, kan^{R}, araC-P_{BAD} | This study |
| | SYNZIP18-szeS_T₂C₃A₃- | |
| | SYNZIP1 and tacI | |
| pNA31 | ori CloDF13, spec^{R}, araC-P_{BAD} | This study |
| | SYNZIP2-szeS_T₃C/E₄A₄T₄ | |
| | C/E₅A₅T₅C₆A₆T₆TE and tacI | |
| pNA34 | ori ColA, kan^{R}, araC-P_{BAD} | This study |
| | SYNZIP18-xtpS_A₃T₃C/ | |
| | E₄A₄T₄-gxpS_TE and tacI | |

1. A protein or a protein fragment comprising at least a first domain or partial domain of a non-ribosomal peptide synthetase (NRPS), a polyketide synthase (PKS) or an NRPS/PKS hybrid synth(et)ase (first PKS-NRPS domain), wherein the protein or the protein fragment has an N-terminus or a C-terminus comprising a first binding domain and wherein this first binding domain preferably represents the N-terminus or C-terminus, respectively, of the protein or the protein fragment, and wherein the first binding domain is characterised by the property of being able to enter into a specific protein-protein binding with at least one corresponding second binding domain.

2. The protein or protein fragment of claim 1, further comprising at least one, preferably two, three or four or more, further PKS-NRPS domain(s), wherein the further PKS-NRPS domain(s) is/are arranged in a direct functional arrangement next to the first PKS-NRPS domain.

3. The protein or protein fragment of any one of claim 1 or 2, wherein the first PKS-NRPS domain, or partial domain, is selected from an A domain, a C domain, a C/E domain, an E domain, a Cₛₜₐᵣₜ domain, an FT domain, or a T domain.

4. The protein or protein fragment according to any one of claims 1 to 3, comprising at least an A domain, a C domain and a T domain, preferably wherein the protein or protein fragment has at least one NRPS-PKS elongation module, an initiation module or a termination module.

5. The protein or protein fragment of any one of claims 1 to 4, wherein the binding domain comprises a synthetic coiled-coil domain (SYNZIP), preferably wherein the SYNZIP is selected from a 1-23 SYNZIP.

6. The protein or protein fragment of any one of the preceding claims, wherein the term opposite the first binding domain comprises a third binding domain, and wherein the third binding domain is characterised by the property of being able to enter into a specific protein-protein binding with at least one corresponding fourth binding domain.

7. The protein or protein fragment of claim 6, wherein the first and second binding domains are selectively capable of binding to the third or fourth binding domain.

8. The protein or protein fragment of any one of claims 1 to 7, wherein the first binding domain is linked to the first PKS-NRPS domain by a linker.

9. An isolated nucleic acid construct comprising a first coding region having a nucleic acid sequence encoding a protein or protein fragment according to any one of claims 1 to 8.

10. A vector system for producing a functional NRPS or PKS, wherein the vector system comprises at least one nucleic acid construct according to claim 9, and wherein the at least one nucleic acid construct is suitable for expressing at least two proteins or protein fragments according to any one of claims 1 to 8, and wherein the at least two proteins or protein fragments are different and together form a functional NRPS, PKS or NRPS/PKS hybrid.

11. The vector system of claim 10, wherein the at least two proteins or protein fragments form the functional NRPS, PKS or NRPS/PKS hybrid through the binding of the first and second binding domains.

12. The vector system according to any one of claim 10 or 11, wherein the vector system comprises nucleic acid constructs suitable for the expression of at least three or more proteins or protein fragments according to any one of claims 1 to 8, or wherein at least two of the three or more proteins or protein fragments together form a functional NRPS, PKS or an NRPS/PKS hybrid.

13. The vector system of claim 23, wherein at least three proteins or protein fragments can together form a functional NRPS, PKS or NRPS/PKS hybrid, wherein the functional NRPS or PKS is formed by binding the proteins or protein fragments to one another by means of binding a first binding domain to a second binding domain and binding a third binding domain to a fourth binding domain.

14. A process for preparing a functional (complete) NRPS or PKS comprising connecting at least a first protein or protein fragment of any one of claims 1 to 8 with a second protein or protein fragment of any one of claims 1 to 8, wherein the first protein or protein fragment has a terminal first binding domain, and wherein the second protein or protein fragment has the terminal second binding domain instead of the terminal first binding domain.


SEQUENCE LISTING (30 sequences)

SEQ ID NO: 1 - protein, 42 aa, Artificial Sequence
  SYNZIP 17
NEKEELKSKKAELRNRIEQLKQKREQLKQKIANLRKEIEAYK

SEQ ID NO: 2 - protein, 42 aa, Artificial Sequence
  SYNZIP 18
SIAATLENDLARLENARLEKDIANLAKLEREEAYEAYEAYEF

SEQ ID NO: 3 - protein, 5 aa, Artificial Sequence
  NRPS connection motif
WNATE

SEQ ID NO: 4 - protein, 4 aa, Artificial Sequence
  Sequence motif
RVLP

SEQ ID NO: 5 - protein, 5 aa, Artificial Sequence
  Connection motif
VYAAP

SEQ ID NO: 6 - protein, 5 aa, Artificial Sequence
  NRPS peptide sequence
SQFLL

SEQ ID NO: 7 - protein, 5 aa, Artificial Sequence
  NRPS peptide sequence
SQLLL

SEQ ID NO: 8 - protein, 5 aa, Artificial Sequence
  NRPS peptide sequence
LTFLL

SEQ ID NO: 9 - protein, 5 aa, Artificial Sequence
  NRPS peptide sequence
LTLLL

SEQ ID NO: 10 - protein, 5 aa, Artificial Sequence
  NRPS peptide sequence
QNFLL

SEQ ID NO: 11 - protein, 5 aa, Artificial Sequence
  NRPS peptide sequence
QNLLL

SEQ ID NO: 12 - protein, 4 aa, Artificial Sequence
  NRPS peptide sequence
VLVV

SEQ ID NO: 13 - protein, 5 aa, Artificial Sequence
  NRPS peptide sequence
VLFLL

SEQ ID NO: 14 - protein, 5 aa, Artificial Sequence
  NRPS peptide sequence
LLFLL

SEQ ID NO: 15 - protein, 5 aa, Artificial Sequence
  NRPS peptide sequence
VLLLL

SEQ ID NO: 16 - protein, 5 aa, Artificial Sequence
  NRPS peptide sequence
LLLLL

SEQ ID NO: 17 - protein, 4 aa, Artificial Sequence
  NRPS peptide sequence
VLFV

SEQ ID NO: 18 - protein, 4 aa, Artificial Sequence
  NRPS peptide sequence
VLLV

SEQ ID NO: 19 - protein, 5 aa, Artificial Sequence
  NRPS peptide sequence
VLVLL

SEQ ID NO: 20 - protein, 5 aa, Artificial Sequence
  NRPS peptide sequence
LLVLL

SEQ ID NO: 21 - protein, 5 aa, Artificial Sequence
  NRPS peptide sequence
VLVLL

SEQ ID NO: 22 - protein, 6 aa, Artificial Sequence
  NRPS peptide sequence
VLVVYW

SEQ ID NO: 23 - protein, 4 aa, Artificial Sequence
  NRPS peptide sequence
VLFV

SEQ ID NO: 24 - protein, 4 aa, Artificial Sequence
  NRPS peptide sequence
LLFV

SEQ ID NO: 25 - protein, 4 aa, Artificial Sequence
  NRPS peptide sequence
VLLV

SEQ ID NO: 26 - protein, 4 aa, Artificial Sequence
  NRPS peptide sequence
LLLV

SEQ ID NO: 27 - protein, 6 aa, Artificial Sequence
  NRPS peptide sequence
VLFVYW

SEQ ID NO: 28 - protein, 6 aa, Artificial Sequence
  NRPS peptide sequence
LLFVYW

SEQ ID NO: 29 - protein, 6 aa, Artificial Sequence
  NRPS peptide sequence
VLLVYW

SEQ ID NO: 30 - protein, 6 aa, Artificial Sequence
  NRPS peptide sequence
LLLVYW